SEQUENCE LISTING (68 sequences)

SEQ ID NO: 1 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 2 - protein, 87 aa, Homo sapiens
VLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 3 - protein, 109 aa, Mus musculus
MRLSTATLLLLLASCLSPGHGILEAHYTNLKCRCSGVISTVVGLNIIDRIQVTPPGNGCPKTEVVIWTKMKKVICVNPRAKWLQRLLRHVQSKSLSSTPQAPVSKRRAA

SEQ ID NO: 4 - protein, 88 aa, Mus musculus
ILEAHYTNLKCRCSGVISTVVGLNIIDRIQVTPPGNGCPKTEVVIWTKMKKVICVNPRAKWLQRLLRHVQSKSLSSTPQAPVSKRRAA

SEQ ID NO: 5 - protein, 122 aa, Artificial Sequence
  Description of Artificial Sequence Synthetic VH XW25-2C4-4H8
QVQLQQPGTELVKPGASVKLSCKASGYNFATYWMHWVKQRPGQGLEWIGNINPSDGGTNFNEKFNNKATLTVDKSSSTAYMQLSSLTSEDSAVYYCAKWGGHYVLYWYFDVWGTGTTVTVSS

SEQ ID NO: 6 - protein, 5 aa, Artificial Sequence
  Description of Artificial Sequence Synthetic VH 2C4 CDR-H1
TYWMH

SEQ ID NO: 7 - protein, 17 aa, Artificial Sequence
  Description of Artificial Sequence Synthetic VH 2C4 CDR-H2
NINPSDGGTNFNEKFNN

SEQ ID NO: 8 - protein, 13 aa, Artificial Sequence
  Description of Artificial Sequence Synthetic VH 2C4 CDR-H3
WGGHYVLYWYFDV

SEQ ID NO: 9 - protein, 108 aa, Artificial Sequence
  Description of Artificial Sequence Synthetic VL XW25-2C4-4H8
DIVLTQSPATLSVTPGDSVSLSCRASQSISSNLHWYQQKSHESPRLLITYASQSISGIPSRFSGSGSGTDFTLSINSVESEDVGVYFCQQSDSWPLTFGAGTKLELKR

SEQ ID NO: 10 - protein, 11 aa, Artificial Sequence
  Description of Artificial Sequence Synthetic VL 2C4 CDR-L1
RASQSISSNLH

SEQ ID NO: 11 - protein, 7 aa, Artificial Sequence
  Description of Artificial Sequence Synthetic VL 2C4 CDR-L2
YASQSIS

SEQ ID NO: 12 - protein, 9 aa, Artificial Sequence
  Description of Artificial Sequence Synthetic VL 2C4 CDR-L3
QQSDSWPLT

SEQ ID NO: 13 - protein, 122 aa, Artificial Sequence
  Description of Artificial Sequence Synthetic VH XW25-3B5-3F4
QVQLQQPGTELVKPGTSVKLSCKASGYTFTSYWMHWVKQRPGQGLEWIGNINPSDGGIKYNEKFKSKATLTVDKSSSTAYMELSSLTSEDSAVYYCAKWGGHYVLYWYFDVWGTGTTVTVSS

SEQ ID NO: 14 - protein, 5 aa, Artificial Sequence
  Description of Artificial Sequence Synthetic VH 3B5 CDR-H1
SYWMH

SEQ ID NO: 15 - protein, 17 aa, Artificial Sequence
  Description of Artificial Sequence Synthetic VH 3B5 CDR-H2
NINPSDGGIKYNEKFKS

SEQ ID NO: 16 - protein, 13 aa, Artificial Sequence
  Description of Artificial Sequence Synthetic VH 3B5 CDR-H3
WGGHYVLYWYFDV

SEQ ID NO: 17 - protein, 108 aa, Artificial Sequence
  Description of Artificial Sequence Synthetic VL XW25-3B5-3F4
DIVLTQSPATLSVTPGDSVSLSCRASQSISNNLHWYQQKSHESPRLLINYASQSISGIPSRFSGSGSGTDFTLSINSVETEDFGMYFCQQSNSWPLTFGAGTKLELKR

SEQ ID NO: 18 - protein, 11 aa, Artificial Sequence
  Description of Artificial Sequence Synthetic VL 3B5 CDR-L1
RASQSISNNLH

SEQ ID NO: 19 - protein, 7 aa, Artificial Sequence
  Description of Artificial Sequence Synthetic VL 3B5 CDR-L2
YASQSIS

SEQ ID NO: 20 - protein, 9 aa, Artificial Sequence
  Description of Artificial Sequence Synthetic VL 3B5 CDR-L3
QQSNSWPLT

SEQ ID NO: 21 - protein, 122 aa, Artificial Sequence
  Description of Artificial Sequence Synthetic VH XW25-4F2-5D2
QVQLQQPGTELVKPGASVKLSCKASGYNFPTYWMHWVKQRPGQGLEWIGNINPSDGGTNFNEKFNNKATLTVDKSSSTAYMQLSSLTSEDSAVYYCAKWGGHYVLYWYFDVWGTGTTVTVSS

SEQ ID NO: 22 - protein, 5 aa, Artificial Sequence
  Description of Artificial Sequence Synthetic VH 4F2 CDR-H1
TYWMH

SEQ ID NO: 23 - protein, 17 aa, Artificial Sequence
  Description of Artificial Sequence Synthetic VH 4F2 CDR-H2
NINPSDGGTNFNEKFNN

SEQ ID NO: 24 - protein, 13 aa, Artificial Sequence
  Description of Artificial Sequence Synthetic VH 4F2 CDR-H3
WGGHYVLYWYFDV

SEQ ID NO: 25 - protein, 108 aa, Artificial Sequence
  Description of Artificial Sequence Synthetic VL XW25-4F2-5D2
DIVLTQSPATLSVTPGDSVSLSCRASQSISNNLHWYQQKSHESPRLLITYASQSISGIPSRFSGSGSGTDFTLSINSVESEDVGVYFCQQSDSWPLTFGAGTKLELKR

SEQ ID NO: 26 - protein, 11 aa, Artificial Sequence
  Description of Artificial Sequence Synthetic VL 4F2 CDR-L1
RASQSISNNLH

SEQ ID NO: 27 - protein, 7 aa, Artificial Sequence
  Description of Artificial Sequence Synthetic VL 4F2 CDR-L2
YASQSIS

SEQ ID NO: 28 - protein, 9 aa, Artificial Sequence
  Description of Artificial Sequence Synthetic VL 4F2 CDR-L3
QQSDSWPLT

SEQ ID NO: 29 - protein, 120 aa, Artificial Sequence
  Description of Artificial Sequence Synthetic VH XW25-5D11-4F3-3D11
EVQVQQSGAELVRPGSSVKMSCKTSGYTFTNYGIIWVKQRPGQGLEWIGYIYIGKGYTEYNEKFKGKATLTSDTSSSTVYMQLSSLTSEDSAIYFCARGAHYYGNAMDYWDQGTSVTVSS

SEQ ID NO: 30 - protein, 5 aa, Artificial Sequence
  Description of Artificial Sequence Synthetic VH 5D11 CDR-H1
NYGII

SEQ ID NO: 31 - protein, 17 aa, Artificial Sequence
  Description of Artificial Sequence Synthetic VH 5D11 CDR-H2
YIYIGKGYTEYNEKFKG

SEQ ID NO: 32 - protein, 11 aa, Artificial Sequence
  Description of Artificial Sequence Synthetic VH 5D11 CDR-H3
GAHYYGNAMDY

SEQ ID NO: 33 - protein, 108 aa, Artificial Sequence
  Description of Artificial Sequence Synthetic VL XW25-5D11-4F3-3D11
DIQMTQTTSSLSASLGDRVTISCRSSQDISNFLNWYQQRPDGTVKLLIYYTSRLHSGVPSRFSGSGSGTDYSLTISNLEQEDIATYFCQQGDTLPWTFGGGTKLEIKR

SEQ ID NO: 34 - protein, 11 aa, Artificial Sequence
  Description of Artificial Sequence Synthetic VL 5D11 CDR-L1
RSSQDISNFLN

SEQ ID NO: 35 - protein, 7 aa, Artificial Sequence
  Description of Artificial Sequence Synthetic VL 5D11 CDR-L2
YTSRLHS

SEQ ID NO: 36 - protein, 9 aa, Artificial Sequence
  Description of Artificial Sequence Synthetic VL 5D11 CDR-L3
QQGDTLPWT

SEQ ID NO: 37 - protein, 122 aa, Artificial Sequence
  Description of Artificial Sequence Synthetic VH XW25-6A2-2F10
QVQLQQPGTELVKPGASVKLSCKASGYNFATYWVHWVKQRPGQGLEWIGNINPSDGGTSYNEKFSKKATLTVDKSSSTAYMELSSLTSEDSAVYYCAKWGGHYVLYWSFDVWGTGTTVTVSS

SEQ ID NO: 38 - protein, 5 aa, Artificial Sequence
  Description of Artificial Sequence Synthetic VH 6A2 CDR-H1
TYWVH

SEQ ID NO: 39 - protein, 17 aa, Artificial Sequence
  Description of Artificial Sequence Synthetic VH 6A2 CDR-H2
NINPSDGGTSYNEKFSK

SEQ ID NO: 40 - protein, 13 aa, Artificial Sequence
  Description of Artificial Sequence Synthetic VH 6A2 CDR-H3
WGGHYVLYWSFDV

SEQ ID NO: 41 - protein, 108 aa, Artificial Sequence
  Description of Artificial Sequence Synthetic VL XW25-6A2-2F10
DIVLTQSPATLSVTPGDSVSLSCRASQSISNNLHWYQQRSHESPRLIITHVSQSISGIPSRFSGSGSGTDFIFSINSVESEDFGVYFCQQSNSWPLTFGAGTKLELKR

SEQ ID NO: 42 - protein, 11 aa, Artificial Sequence
  Description of Artificial Sequence Synthetic VL 6A2 CDR-L1
RASQSISNNLH

SEQ ID NO: 43 - protein, 7 aa, Artificial Sequence
  Description of Artificial Sequence Synthetic VL 6A2 CDR-L2
HVSQSIS

SEQ ID NO: 44 - protein, 9 aa, Artificial Sequence
  Description of Artificial Sequence Synthetic VL 6A2 CDR-L3
QQSNSWPLT

SEQ ID NO: 45 - protein, 122 aa, Artificial Sequence
  Description of Artificial Sequence Synthetic VH XW25-7C7-5E5
QVQLQQPGTELVKPGASVKLSCKASGYNFATYWMHWVKQRPGQGLEWIGNINPSDGGTNFNEKFNNKATLTVDKSSSTAYMHLSSLTSEDSAVYYCAKWGGHYVLYWYFDVWGTGTTVTVSS

SEQ ID NO: 46 - protein, 5 aa, Artificial Sequence
  Description of Artificial Sequence Synthetic VH 7C7 CDR-H1
TYWMH

SEQ ID NO: 47 - protein, 17 aa, Artificial Sequence
  Description of Artificial Sequence Synthetic VH 7C7 CDR-H2
NINPSDGGTNFNEKFNN

SEQ ID NO: 48 - protein, 13 aa, Artificial Sequence
  Description of Artificial Sequence Synthetic VH 7C7 CDR-H3
WGGHYVLYWYFDV

SEQ ID NO: 49 - protein, 108 aa, Artificial Sequence
  Description of Artificial Sequence Synthetic VL XW25-7C7-5E5
DIVLTQSPATLSVTPGDSVSLSCRASQNINNNLHWYQQKSHESPRLLITYTSQSISGIPSRFSGSGSGTDFTLSINSVESEDVGVYFCQQSDSWPLTFGAGTKLELKR

SEQ ID NO: 50 - protein, 11 aa, Artificial Sequence
  Description of Artificial Sequence Synthetic VL 7C7 CDR-L1
RASQNINNNLH

SEQ ID NO: 51 - protein, 7 aa, Artificial Sequence
  Description of Artificial Sequence Synthetic VL 7C7 CDR-L2
YTSQSIS

SEQ ID NO: 52 - protein, 9 aa, Artificial Sequence
  Description of Artificial Sequence Synthetic VL 7C7 CDR-L3
QQSDSWPLT

SEQ ID NO: 53 - protein, 122 aa, Artificial Sequence
  Description of Artificial Sequence Synthetic VH XW25-8H6-4F9
QVQLQQPGTELVKPGASVKLSCKASGYNFATYWMHWVKQRPGQGLEWIGNINPSDGGTNYNEKFNNKATLTVDKSSSTAYMQLSSLTSEDSAVYYCAKWGGHYVLYWYFDVWGTGTTVTVSS

SEQ ID NO: 54 - protein, 5 aa, Artificial Sequence
  Description of Artificial Sequence Synthetic VH 8H6 CDR-H1
TYWMH

SEQ ID NO: 55 - protein, 17 aa, Artificial Sequence
  Description of Artificial Sequence Synthetic VH 8H6 CDR-H2
NINPSDGGTNYNEKFNN

SEQ ID NO: 56 - protein, 13 aa, Artificial Sequence
  Description of Artificial Sequence Synthetic VH 8H6 CDR-H3
WGGHYVLYWYFDV

SEQ ID NO: 57 - protein, 108 aa, Artificial Sequence
  Description of Artificial Sequence Synthetic VL XW25-8H6-4F9
DIVLTQSPATLSVTPGDSVSLSCRASQSINNNLHWYQQESHESPRLLITYASQSISGIPSRFSGSGSGTDFTLSINSVESEDFGVYFCQQSNSWPLTFGAGTKLELKR

SEQ ID NO: 58 - protein, 11 aa, Artificial Sequence
  Description of Artificial Sequence Synthetic VL 8H6 CDR-L1
RASQSINNNLH

SEQ ID NO: 59 - protein, 7 aa, Artificial Sequence
  Description of Artificial Sequence Synthetic VL 8H6 CDR-L2
YASQSIS

SEQ ID NO: 60 - protein, 9 aa, Artificial Sequence
  Description of Artificial Sequence Synthetic VL 8H6 CDR-L3
QQSNSWPLT

SEQ ID NO: 61 - protein, 122 aa, Artificial Sequence
  Description of Artificial Sequence Synthetic VH XW25-9C10-4B11
QVQLQQPGTELVKPGASVKLSCKASGYNFATYWMHWVKQRPGQGLEWIGNINPSDGGTNFNEKFNSKATLTVDKSSSTAYMQLSSLTSEDSAVYYCAKWGGHYVLYWYFDVWGTGTTVTVSS

SEQ ID NO: 62 - protein, 5 aa, Artificial Sequence
  Description of Artificial Sequence Synthetic VH 9C10 CDR-H1
TYWMH

SEQ ID NO: 63 - protein, 17 aa, Artificial Sequence
  Description of Artificial Sequence Synthetic VH 9C10 CDR-H2
NINPSDGGTNFNEKFNS

SEQ ID NO: 64 - protein, 13 aa, Artificial Sequence
  Description of Artificial Sequence Synthetic VH 9C10 CDR-H3
WGGHYVLYWYFDV

SEQ ID NO: 65 - protein, 108 aa, Artificial Sequence; identical to SEQ ID NO: 9
  Description of Artificial Sequence Synthetic VL XW25-9C10-4B11

SEQ ID NO: 66 - protein, 11 aa, Artificial Sequence
  Description of Artificial Sequence Synthetic VL 9C10 CDR-L1
RASQSISSNLH

SEQ ID NO: 67 - protein, 7 aa, Artificial Sequence
  Description of Artificial Sequence Synthetic VL 9C10 CDR-L2
YASQSIS

SEQ ID NO: 68 - protein, 9 aa, Artificial Sequence
  Description of Artificial Sequence Synthetic VL 9C10 CDR-L3
QQSDSWPLT

(19) United States
(12) Patent Application Publication
(10) Pub. No.: US 2008/0227704 A1
(43) Pub. Date: Sep. 18, 2008

(54) CXCL13 binding proteins

(76) Inventor: Joanne S. Kamens, Newton Center, MA (US)

Correspondence Address: ABBOTT BIORESEARCH, 100 RESEARCH DRIVE, WORCESTER, MA, 01605-4314, US

(21) Appl. No.: 12/004,677

(22) Filed: Dec. 20, 2007

Related U.S. Application Data
(60) Provisional application No. 60/876,375, filed on Dec. 21, 2006.

(51) Int. Cl. A61K 38/00; C07K 14/00; C07K 7/00; C07K 16/18; C12N 15/11; C12N 15/00; A01K 67/027; A61P 43/00; C12N 5/06; C12N 1/20; C12N 5/04; C12N 1/16; C12P 21/04

(52) U.S. Cl. 514/12; 530/350; 530/328; 530/387.3; 530/391.3; 530/391.7; 536/23.1; 536/23.53; 435/320.1; 435/325; 435/252.3; 435/252.33; 435/419; 435/254.11; 435/348; 435/349; 435/358; 435/254.21; 435/69.6; 800/3

(57) ABSTRACT

The present invention encompasses CXCL13 binding proteins. Specifically, the invention relates to antibodies that are wild-type, chimeric, CDR grafted and humanized. Preferred antibodies have high affinity for CXCL13 and CXCL13 activity in vitro and in vivo. An antibody of the invention can be a full-length antibody or an antigen-binding portion thereof. Methods of making and methods of using the antibodies of the invention are also provided. The antibodies, or antigen-binding portions, of the invention are useful for detecting mouse CXCL13 and for inhibiting mouse CXCL13 activity, e.g., in a mammal suffering from a disorder in which CXCL13 activity is detrimental.

### CROSS REFERENCE TO RELATED APPLICATION

This application claims priority to U.S. Provisional Application Ser. No. 60/876,375 filed on Dec. 21, 2006.

### FIELD OF THE INVENTION

The present invention relates to CXCL13 binding proteins, and specifically to their uses.

### BACKGROUND OF THE INVENTION

CXCL13 (BLC, BCA1, ANGIE, BCA-1, BLR1L, ANGIE2, SCYB13) is a chemokine expressed in follicular stromal cells of lymphoid organs (Ansel, et al. Nature 406, 309 (2000)), macrophages in the peritoneal and pleural cavities (Ansel, et al. Immunity 16, 67 (2002)) and in myeloid dendritic cells (Ishikawa, et al. J. Exp. Med. 193, 1393 (2001)). It has been shown to bind primarily to the G-protein coupled receptor CXCR5 (Gunn, et al. Nature 391, 799 (1998)). CXCR5 is expressed on B cells and certain subsets of T cells (including follicular helper T cells, a subset of circulating memory CD4 T cells, and other populations of T cells not fully differentiated as Th1 or Th2) (Chtanova, et al. J. Immunol. 173, 68 (2004); Kim, et al. J. Exp. Med. 193, 1373 (2001); Lim, et al. J. Clin. Invest. 114, 1640 (2004)). CXCL13 has demonstrated physiologic roles in co-localization of B and T cells by influencing homing of auto-reactive B1 cells to Peyer's patches and other sites of inflammation (Ishikawa, et al. J. Exp. Med. 193, 1393 (2001)), and by playing a role in recruitment of Th cells to secondary lymphoid organs for T dependent antibody production (Kim, et al. J. Exp. Med. 193, 1373 (2001); Ebert, et al. Eur. J. Immunol. 34, 3562 (2004); Moser, et al. Trends Immunol. 23, 250 (2002); Vissers, et al. Eur. J. Immunol. 31, 1544 (2001)). The CXCL13 KO mouse phenotype has been published and has been shown to be similar to the CXCR5 KO phenotype (Ansel, et al. Immunity 16, 67 (2002)). These studies have demonstrated that CXCL13 is required for development of most lymph nodes and Peyer's patches although the phenotype is not completely penetrant. For example, 57% of CXCL13 KO mice have no Peyer's patch development along the small intestine and the remaining 43% have 1-6 patches. In comparison, 93% of wild type animals have >6 Peyer's patches along the small intestine.

Inhibition of CXCL13 activity by an antibody reagent could inhibit immune disease progression by at least three possible mechanisms of action. First, blocking CXCL13 activity could prevent migration of B cells (especially B1 cells) to relevant target organs such as CNS for MS or kidney for SLE. CXCL13 would be the first target to address a B1 cell mediated mechanism of action. Second, literature strongly supports a requirement for CXCL13 in the formation of germinal centers (GCs) via its role in B and T cell localization. Therefore, inappropriate formation of GCs at disease sites could be targeted via an anti-CXCL13 blockade. It has been observed that inappropriate GCs and follicular structures can be observed in affected target organs in a variety of different immune diseases. Finally, preventing proper localization of B and T cells might result in the effective prevention of co-stimulation and reduction of T cell activation. This may be especially important in secondary immune responses where B cells may have a significant antigen presentation function.

There is a need in the art for improved antibodies capable of binding CXCL13. Preferably the antibodies bind mouse CXCL13. Preferably the antibodies are capable of neutralizing mouse CXCL13. The present invention provides a novel family of binding proteins, and fragments thereof, capable binding mouse CXCL13, binding with high affinity, and binding and neutralizing mouse CXCL13.

### SUMMARY OF THE INVENTION

In one aspect, the invention relates to isolated binding proteins, or fragments thereof, that bind mouse CXCL13. In an embodiment, the isolated binding proteins, or fragments thereof, are capable of modulating a biological function of mouse CXCL13, for example, by neutralizing mouse CXCL13. In yet another embodiment, the isolated binding proteins, or fragments thereof, are capable of preventing the binding of mouse CXCL13 to the mouse CXCL13 receptor CXCL5.

In an embodiment, isolated binding protein, or fragment thereof, binds mouse CXCL13 with an EC₅₀ of less than about 12.0 nM using an ELISA test. In another embodiment, the isolated binding protein, or fragment thereof inhibits calcium influx induced by mouse CXCL13 to the mouse CXCL13 receptor CXCL5 in a CXCL5 receptor mediated FLIPR assay with an average Kb of about 65 to about 95. In still a further embodiment, the isolated binding protein has been affinity maturated.

In an embodiment, the invention provides an isolated binding protein, or fragment thereof, comprising an antigen binding domain capable of binding mouse CXCL13, comprising at least one CDR having an amino acid sequence selected from the group consisting of SEQ ID NOs: 6, 7, 8, 10, 11, 12, 14, 15, 16, 18, 19, 20, 22, 23, 24, 26, 27, 28, 30, 31, 32, 34, 35, 36, 38, 39, 40, 42, 43, 44, 46, 47, 48, 50, 51, 52, 54, 55, 56, 58, 59, 60, 62, 63, 64, 66, 67, and 68.

In an embodiment, the invention provides an isolated binding protein comprising an antigen binding domain, wherein the binding protein is capable of binding mouse CXCL13, and wherein the antigen binding domain comprises at least one VH region comprising an amino acid sequence selected from the group consisting of SEQ ID NOs: 5, 13, 21, 29, 37, 45, 53, and 61. In still a further embodiment, the invention provides an isolated binding protein comprising an antigen binding domain, wherein the binding protein is capable of binding mouse CXCL13, and wherein the antigen binding domain comprises at least one VL region comprising an amino acid sequence selected from the group consisting of SEQ ID NOs: 9, 17, 25, 33, 41, 49, 57, and 65. For example, the isolated binding protein comprises at least 3 CDRs, such as, for example, SEQ ID NOs: 6, 7, and 8; SEQ ID NOs: 14, 15, and 16; SEQ ID NOs: 22, 23, and 24; SEQ ID NOs: 30, 31, and 32; SEQ ID NOs: 38, 39, and 40; and SEQ ID NOs: 46, 47, and 48; SEQ ID NOs: 54, 55, and 56; and SEQ ID NOs: 62, 63, and 64. In another embodiment, the at least 3 CDRs are selected from a VL CDR set selected from the group consisting of SEQ ID NOs: 10, 11, and 12; SEQ ID NOs: 18, 19, and 20; SEQ ID NOs: 26, 27, and 28; SEQ ID NOs: 34, 35, and 36; SEQ ID NOs: 42, 43, and 44; and SEQ ID NOs: 50, 51, and 52; SEQ ID NOs: 58, 59, and 60; and SEQ ID NOs: 66, 67, and 68.

In another embodiment, the isolated binding protein comprises at least two variable domain CDR sets, for example, SEQ ID NOs: 6, 7, 8 and SEQ ID NOs: 10, 11, 12; SEQ ID NOs: 14, 15 16 and SEQ ID NOs: 18, 19, 20; SEQ ID NOs: 22, 23, 24 and SEQ ID NOs: 26, 27, 28; SEQ ID NOs: 30, 31, 32 and SEQ ID NOs: 34, 35, 36; SEQ ID NOs: 38, 39, 40 and SEQ ID NOs: 42, 43, 44; SEQ ID NOs: 46, 47, 48 and SEQ ID NOs: 50, 51, 52; SEQ ID NOs: 54, 55, 56 and SEQ ID NOs: 58, 59, 60; and SEQ ID NOs: 62, 63, 64 and SEQ ID NOs: 66, 67, 68.

In another embodiment, the binding protein comprises two variable domains, wherein the two variable domains have amino acid sequences selected from the group consisting of SEQ ID NO:5 and SEQ ID NO:9; SEQ ID NO:13 and SEQ ID NO: 17; SEQ ID NO:21 and SEQ ID NO:25; SEQ ID NO:29 and SEQ ID NO:33; SEQ ID NO:37 and SEQ ID NO:41; SEQ ID NO:45 and SEQ ID NO:49; SEQ ID NO:53 and SEQ ID NO:57; and SEQ ID NO:61 and SEQ ID NO:65.

In another embodiment, the isolated binding protein binds a protein comprising the sequence of SEQ ID NO: 3 or SEQ ID NO:4.

In another aspect, the invention provides an antibody construct comprising a binding protein that binds mouse CXCL13, and a linker polypeptide or an immunoglobulin constant domain. The binding protein is selected from the group consisting of an immunoglobulin molecule, a disulfide linked Fv, a monoclonal antibody, a scFv, a chimeric antibody, a single domain antibody, a CDR-grafted antibody, a diabody, a humanized antibody, a multispecific antibody, a Fab, a dual specific antibody, and a Fab′, a bispecific antibody, a F(ab′)2, and a Fv.

In an embodiment, the binding protein comprises a heavy chain immunoglobulin constant domain selected from the group consisting of a human IgM constant domain, a human IgG4 constant domain, a human IgG1 constant domain, a human IgE constant domain, a human IgG2 constant domain, and a human IgG3 constant domain, and a human IgA constant domain.

In an embodiment, the binding protein possesses a human glycosylation pattern.

In another embodiment, the binding protein is a crystallized antibody construct, such as a carrier-free pharmaceutical controlled release crystallized antibody construct. Preferably, the antibody construct has a greater half life in vivo than the soluble counterpart of said antibody construct, and retains it biological activity.

In another embodiment, the invention provides an antibody conjugate that comprises an agent selected from the group consisting of an immunoadhension molecule, an imaging agent, a therapeutic agent, and a cytotoxic agent. In an embodiment, the agent is an imaging agent selected from the group consisting of a radiolabel, an enzyme, a fluorescent label, a luminescent label, a bioluminescent label, a magnetic label, and biotin. Exemplary imaging agents are radiolabels selected from the group consisting of: ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm.

In another embodiment, the agent is a therapeutic or cytotoxic agent selected from the group consisting of an anti-metabolite, an alkylating agent, an antibiotic, a growth factor, a cytokine, an anti-angiogenic agent, an anti-mitotic agent, an anthracycline, toxin, and an apoptotic agent.

In another aspect, the invention provides an isolated nucleic acid encoding a binding protein amino acid sequence of CXCL13 and vectors to which they are operably linked, such as pcDNA, pTT, pTT3, pEFBOS, pBV, pJV, and pBJ.

In another aspect, the invention provides a host cell comprising the vector of the invention. The host cell may be a prokaryotic cell, such as E. coli. Alternatively, the host cell is a eukaryotic cell, such as, for example, a protist cell, animal cell, plant cell, a fungal cell, a mammalian cell, an avian cell, and an insect cell. In an embodiment, the host cell is a CHO cell, a COS cell, a yeast cell (such as Saccharomyces cerevisiae), or an Sf9 insect cell.

In another aspect, the invention provides a method of producing a protein capable of binding mouse CXCL13, the method comprising the steps of culturing a host cell of the invention in culture medium under conditions sufficient to produce a binding protein capable of binding mouse CXCL13. The invention also provides a protein produced according to the method. In another embodiment, the invention provides a method for reducing CXCL13 activity in a mammal, comprising contacting CXCL13 with the binding protein of the invention such that CXCL13 activity is reduced. In another embodiment, the invention provides a method for reducing CXCL13 activity in a mammal suffering from a disorder in which CXCL13 activity is detrimental, comprising administering to the mammal the binding protein of the invention such that CXCL13 activity in the mammal is reduced. In another embodiment, the invention provides a method for treating a mammal for a disease or a disorder in which CXCL13 activity is detrimental by administering to the mammal the binding protein of the invention such that treatment is achieved.

Disorders that may be treated with the binding protein of the invention include respiratory disorders; asthma; allergic and nonallergic asthma; asthma due to infection; asthma due to infection with respiratory syncytial virus (RSV); chronic obstructive pulmonary disease (COPD); other conditions involving airway inflammation; eosinophilia; fibrosis and excess mucus production; cystic fibrosis; pulmonary fibrosis; atopic disorders; atopic dermatitis; urticaria; eczema; allergic rhinitis; and allergic enterogastritis; inflammatory and/or autoimmune conditions of the skin; inflammatory and/or autoimmune conditions of gastrointestinal organs; inflammatory bowel diseases (IBD); ulcerative colitis; Crohn's disease; inflammatory and/or autoimmune conditions of the liver; liver cirrhosis; liver fibrosis; liver fibrosis caused by hepatitis B and/or C virus; scleroderma; tumors or cancers; hepatocellular carcinoma; glioblastoma; lymphoma; Hodgkin's lymphoma; viral infections; HTLV-1 infection (e.g., from HTLV-1); suppression of expression of protective type 1 immune responses, and suppression of expression of protective type I immune responses during vaccination, further rheumatoid arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dernatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjörgren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma), Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aordic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, Burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chromic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetes, diabetes mellitus, diabetic ateriosclerotic disease, Diffuse Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, epstein-barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallerrorden-Spatz disease, hashimoto's thyroiditis, hay fever, heart transplant rejection, hemachromatosis, hemodialysis, hemolytic uremic syndrome/thrombolytic thrombocytopenic purpura, hemorrhage, hepatitis (A), H is bundle arrythmias, HIV infection/HIV neuropathy, Hodgkin's disease, hyperkinetic movement disorders, hypersensitivity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic neuritis, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphederma, malaria, malignamt Lymphoma, malignant histiocytosis, malignant melanoma, meningitis, meningococcemia, metabolic/idiopathic, migraine headache, mitochondrial multi.system disorder, mixed connective tissue disease, monoclonal gammopathy, multiple myeloma, multiple systems degenerations (Mencel Dejerine-Thomas Shi-Drager and Machado-Joseph), myasthenia gravis, mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occulsive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherloscierotic disease, peripheral vascular disorders, peritonitis, pernicious anemia, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, Progressive supranucleo Palsy, primary pulmonary hypertension, radiation therapy, Raynaud's phenomenon and disease, Raynoud's disease, Refsum's disease, regular narrow QRS tachycardia, renovascular hypertension, reperfusion injury, restrictive cardiomyopathy, sarcomas, scleroderma, senile chorea, Senile Dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, Subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, Acute coronary syndromes, Acute Idiopathic Polyneuritis, Acute Inflammatory Demyelinating Polyradiculoneuropathy, Acute ischemia, Adult Still's Disease, Alopecia greata, Anaphylaxis, Anti-Phospholipid Antibody Syndrome, Aplastic anemia, Arteriosclerosis, Atopic eczema, Atopic dermatitis, Autoimmune dermatitis, Autoimmune disorder associated with Streptococcus infection, Autoimmune Enteropathy, Autoimmune hearingloss, Autoimmune Lymphoproliferative Syndrome (ALPS), Autoimmune myocarditis, Autoimmune premature ovarian failure, Blepharitis, Bronchiectasis, Bullous pemphigoid, Cardiovascular Disease, Catastrophic Antiphospholipid Syndrome, Celiac Disease, Cervical Spondylosis, Chronic ischemia, Cicatricial pemphigoid, Clinically isolated Syndrome (CIS) with Risk for Multiple Sclerosis, Conjunctivitis, Childhood Onset Psychiatric Disorder, Chronic obstructive pulmonary disease (COPD), Dacryocystitis, dermatomyositis, Diabetic retinopathy, Diabetes mellitus, Disk herniation, Disk prolaps, Drug induced immune hemolytic anemia, Endocarditis, Endometriosis, endophthalmitis, Episcleritis, Erythema multiforme, erythema multiforme major, Gestational pemphigoid, Guillain-Barré Syndrome (GBS), Hay Fever, Hughes Syndrome, Idiopathic Parkinson's Disease, idiopathic interstitial pneumonia, IgE-mediated Allergy, Immune hemolytic anemia, Inclusion Body Myositis, Infectious ocular inflammatory disease, Inflammatory demyelinating disease, Inflammatory heart disease, Inflammatory kidney disease, IPF/UIP, Iritis, Keratitis, Keratojuntivitis sicca, Kussmaul disease or Kussmaul-Meier Disease, Landry's Paralysis, Langerhan's Cell Histiocytosis, Livedo reticularis, Macular Degeneration, Microscopic Polyangiitis, Morbus Bechterev, Motor Neuron Disorders, Mucous membrane pemphigoid, Multiple Organ failure, Myasthenia Gravis, Myelodysplastic Syndrome, Myocarditis, Nerve Root Disorders, Neuropathy, Non-A Non-B Hepatitis, Optic Neuritis, Osteolysis, Pauciarticular JRA, peripheral artery occlusive disease (PAOD), peripheral vascular disease (PVD), peripheral artery disease (PAD), Phlebitis, Polyarteritis nodosa (or periarteritis nodosa), Polychondritis, Polymyalgia Rheumatica, Poliosis, Polyarticular JRA, Polyendocrine Deficiency Syndrome, Polymyositis, polymyalgia rheumatica (PMR), Post-Pump Syndrome, primary parkinsonism, Prostatitis, Pure red cell aplasia, Primary Adrenal Insufficiency, Recurrent Neuromyelitis Optica, Restenosis, Rheumatic heart disease, SAPHO (synovitis, acne, pustulosis, hyperostosis, and osteitis), Scleroderma, Secondary Amyloidosis, Shock lung, Scleritis, Sciatica, Secondary Adrenal Insufficiency, Silicone associated connective tissue disease, Sneddon-Wilkinson Dermatosis, spondilitis ankylosans, Stevens-Johnson Syndrome (SJS), Systemic inflammatory response syndrome, Temporal arthritis, toxoplasmic retinitis, toxic epidermal necrolysis, Transverse myelitis, TRAPS (Tumor Necrosis Factor Receptor, Type 1 allergic reaction, Type II Diabetes, Urticaria, Usual interstitial pneumonia (UIP), Vasculitis, Vernal conjunctivitis, viral retinitis, Vogt-Koyanagi-Harada syndrome (VKH syndrome), Wet macular degeneration, and Wound healing.

The invention provides methods of treating a mammal suffering from a disorder in which CXCL13 is detrimental, the method comprising the step of administering the binding protein of the invention before, concurrent, or after the administration of a second agent, wherein the second agent is selected from the group consisting of inhaled steroids; beta-agonists; short-acting or long-acting beta-agonists; antagonists of leukotrienes or leukotriene receptors; ADVAIR; IgE inhibitors; anti-IgE antibodies; XOLAIR; phosphodiesterase inhibitors; PDE4 inhibitors; xanthines; anticholinergic drugs; mast cell-stabilizing agents; Cromolyn; IL-4 inhibitors; IL-5 inhibitors; eotaxin/CCR3 inhibitors; antagonists of histamine or its receptors including H1, H2, H3, and H4; antagonists of prostaglandin D or its receptors DP1 and CRTH2; TNF antagonists; a soluble fragment of a TNF receptor; ENBREL; TNF enzyme antagonists; TNF converting enzyme (TACE) inhibitors; muscarinic receptor antagonists; TGF-beta antagonists; interferon gamma; perfenidone; chemotherapeutic agents, methotrexate; leflunomide; sirolimus (rapamycin) or an analog thereof, CCI-779; COX2 or cPLA2 inhibitors; NSAIDs; immunomodulators; p38 inhibitors; TPL-2, MK-2 and NFkB inhibitors; budenoside; epidermal growth factor; corticosteroids; cyclosporine; sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β antibodies; anti-IL-6 antibodies; growth factors; elastase inhibitors; pyridinyl-imidazole compounds; antibodies or agonists of TNF, LT, IL-1, IL-2, IL-3, IL-4, IL-5, IL-6, IL-7, IL-8, IL-9, IL-10, IL-11, IL-12, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, EMAP-II, GM-CSF, FGF, or PDGF; antibodies of CD2, CD3, CD4, CD8, CD25, CD28, CD30, CD40, CD45, CD69, CD90 or their ligands; FK506; rapamycin; mycophenolate mofetil; ibuprofen; prednisolone; phosphodiesterase inhibitors; adenosine agonists; antithrombotic agents; complement inhibitors; adrenergic agents; IRAK, NIK, IKK, p38, or MAP kinase inhibitors; IL-1β converting enzyme inhibitors; TNFα converting enzyme inhibitors; T-cell signaling inhibitors; metalloproteinase inhibitors; 6-mercaptopurines; angiotensin converting enzyme inhibitors; soluble cytokine receptors; soluble p55 TNF receptor; soluble p75 TNF receptor; sIL-1RI; sIL-1RII; sIL-6R; anti-inflammatory cytokines; IL-4; IL-10; IL-11; and TGFβ.

In invention comprises administering to a subject is by at least one mode selected from parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal.

In another aspect, the invention provides an isolated antibody, or antigen binding fragment thereof, wherein said antibody, or antigen binding fragment thereof binds mouse CXCL13 and inhibits the binding of CXCL13 to the CXCR5 receptor in a cell surface-based receptor binding assay with an IC₅₀ selected from the group consisting of less than about 12.0 nM using an ELISA test. In an embodiment, the isolated binding protein, or fragment thereof inhibits calcium influx induced by the mouse CXCL13 to CXCL5 in a CXCL5 receptor mediated FLIPR assay with an average Kb of about 65 to about 95.

In an embodiment, the antibody is selected from the group consisting of 5D11, 2C4, 6A2, 4F2, 7C7, 8H6, 9C10, and 3B5.

The invention further provides an isolated antibody, or antigen binding fragment thereof, that binds mouse CXCL13 with binding characteristics selected from the group consisting of an on rate constant (kₒₙ) between about 1.0×10⁶M⁻¹s⁻¹ to about 3×10⁶M⁻¹ s⁻¹; an off rate constant (k_{off}) of about 3.0×10⁻⁴ s⁻¹ to about 3.0×10⁻⁶ s⁻¹; as measured by surface plasmon resonance; and a dissociation constant (K_{D}) of about 2 μM to about 13 pM. In an embodiment, the antibody, or antigen binding fragment thereof, has a dissociation constant (K_{D}) to mouse CXCL13 selected from the group consisting of about 2.0 μM, about 3.0 μM, and about 13.0 μM.

In an embodiment, the antibody, or antigen binding fragment thereof is capable of modulating a biological function of mouse CXCL13, for example, neutralizing mouse CXCL13.

In another aspect, the invention relates to a method of generating an antibody, or fragment thereof, that binds to mouse CXCL13, comprising the steps of immunizing a non-human animal with CXCL13; collecting a body fluid or organ comprising an anti-CXCL13 antibody; and isolating said anti-CXCL13 antibody.

### DETAILED DESCRIPTION OF THE INVENTION

This invention pertains to CXCL13 binding proteins, particularly anti-mouse CXCL13 antibodies, or antigen-binding portions thereof, that bind mouse CXCL13. Various aspects of the invention relate to antibodies and antibody fragments, as well as nucleic acids, recombinant expression vectors and host cells for making such antibodies and fragments. Methods of using the antibodies of the invention to detect mouse CXCL13; to inhibit mouse CXCL13 activity, either in vitro or in vivo; and to regulate gene expression are also encompassed by the invention.

Unless otherwise defined herein, scientific and technical terms used in connection with the present invention shall have the meanings that are commonly understood by those of ordinary skill in the art. The meaning and scope of the terms should be clear, however, in the event of any latent ambiguiy, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques described herein are those well known and commonly used in the art.

That the present invention may be more readily understood, select terms are defined below.

The term “polypeptide” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein sequence. A polypeptide may be monomeric or polymeric.

The term “isolated protein” or “isolated polypeptide” is a protein or polypeptide that by virtue of its origin or source of derivation is not associated with naturally associated components that accompany it in its native state; is substantially free of other proteins from the same species; is expressed by a cell from a different species; or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “recovering” as used herein, refers to the process of rendering a chemical species such as a polypeptide substantially free of naturally associated components by isolation, e.g., using protein purification techniques well known in the art.

The term “CXCL13” as used herein, includes CXCL13, BLC, BCA1, ANGIE, BCA-1, BLR1L, ANGIE2, SCYB13 and is a chemokine expressed in follicular stromal cells of lymphoid organs, macrophages in the peritoneal and pleural cavities and in myeloid dendritic cells. It has been shown to bind primarily to the G-protein coupled receptor CXCR5. The term mouse CXCL13 is intended to include recombinant mouse CXCL13 (rh mouse CXCL13), which can be prepared by standard recombinant expression methods.

Table 1 shows the amino acid sequences of human and mouse CXCL13, and their respective secreted forms, all of which are known in the art.

TABLE 1
Sequence of CXCL13

| | Sequence | Sequence |
|---|---|---|
| Protein | Identifier | 12345678901234567890123456789012 |

| | | |
|---|---|---|
| Human CXCL13 | SEQ ID NO.:1 | MKFISTSLLLMLLVSSLSPVQGVLEVYYTSLR |
| | | CRCVQESSVFIPRRFIDRIQILPRGNGCPRKE |
| | | IIVWKKNKSIVCVDPQAEWIQRMMEVLRKRSS |
| | | STLPVPVFKRKIP |
| Secreted form of | SEQ ID NO.:2 | VLEVYYTSLRCRCVQESSVFIPRRFIDRIQIL |
| Human CXCL13 | | PRGNGCPRKEIIVWKKNKSIVCVDPQAEWIQR |
| | | MMEVLRKRSSSTLPVPVFKRKIP |
| Mouse CXCL13 | SEQ ID NO.:3 | MRLSTATLLLLLASCLSPGHGILEAHYTNLKC |
| | | RCSGVISTVVGLNIIDRIQVTPPGNGCPKTEV |
| | | VIWTKMKKVICVNPRAKWLQRLLRHVQSKSLS |
| | | STPQAPVSKRRAA |
| Secreted form of | SEQ ID NO.:4 | ILEAHYTNLKCRCSGVISTVVGLNIIDRIQVT |
| Mouse CXCL13 | | PPGNGCPKTEVVIWTKMKKVICVNPRAKWLQR |
| | | LLRHVQSKSLSSTPQAPVSKRRAA |

“Biological activity” as used herein, refers to all inherent biological properties of the cytokine. Biological properties of CXCL13 include but are not limited to physiologic roles in co-localization of B and T cells by influencing homing of auto-reactive B1 cells to Peyer's patches and other sites of inflammation, and playing a role in recruitment of Th cells to secondary lymphoid organs for T dependent antibody production.

The terms “specific binding” or “specifically binding”, as used herein, in reference to the interaction of an antibody, a protein, or a peptide with a second chemical species, mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Nonlimiting embodiments of which are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass.

The term “antigen-binding portion” of an antibody (or simply “antibody portion”), as used herein, refers to one or more fragments of an antibody that retain the ability to specifically bind to an antigen (e.g., mouse CXCL13). It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-54041354-5).

The term “antibody construct” as used herein refers to a polypeptide comprising one or more the antigen binding portions of the invention linked to a linker polypeptide or an immunoglobulin constant domain. Linker polypeptides comprise two or more amino acid residues joined by peptide bonds and are used to link one or more antigen binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). An immunoglobulin constant domain refers to a heavy or light chain constant domain.

Still further, an antibody or antigen-binding portion thereof may be part of a larger immunoadhesion molecules, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

An “isolated antibody”, as used herein, is intended to refer to an antibody that is substantially free of other antibodies having different antigenic specificities (e.g., an isolated antibody that specifically binds mouse CXCL13 is substantially free of antibodies that specifically bind antigens other than mouse CXCL13). An isolated antibody that specifically binds mouse CXCL13 may, however, have cross-reactivity to other antigens, such as CXCL13 molecules from other species. Moreover, an isolated antibody may be substantially free of other cellular material and/or chemicals.

The term “chimeric antibody” refers to antibodies which comprise heavy and light chain variable region sequences from one species and constant region sequences from another species, such as antibodies having murine heavy and light chain variable regions linked to human constant regions.

The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “humanized antibody” refers to antibodies which comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences.

As used herein, the term “neutralizing” refers to neutralization of biological activity of a chemokine when a binding protein specifically binds the chemokine. Preferably a neutralizing binding protein is a neutralizing antibody whose binding to mouse CXCL13 results in inhibition of a biological activity of mouse CXCL13. Preferably the neutralizing binding protein binds mouse CXCL13 and reduces a biologically activity of mouse CXCL13 by at least about 20%, 40%, 60%, 80%, 85% or more. Inhibition of a biological activity of mouse CXCL13 by a neutralizing binding protein can be assessed by measuring one or more indicators of mouse CXCL13 biological activity well known in the art. For example inhibition of chemotaxis of recombinant BA/F3 murine CXCR5 cells towards purified mouse CXCL13 (see Example 1.1.C).

The term “activity” includes activities such as the binding specificity/affinity of an antibody for an antigen, for example, an anti-mouse CXCL13 antibody that binds to a mouse CXCL13 antigen and/or the neutralizing potency of an antibody, for example, an anti-mouse CXCL13 antibody whose binding to mouse CXCL13 inhibits the biological activity of mouse CXCL13, as described in Example 1.1.C.

The term “epitope” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jönsson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “Kₒₙ”, as used herein, is intended to refer to the on rate constant for association of an antibody to the antigen to form the antibody/antigen complex as is known in the art.

The term “K_{off}”, as used herein, is intended to refer to the off rate constant for dissociation of an antibody from the antibody/antigen complex as is known in the art.

The term “K_{d}”, as used herein, is intended to refer to the dissociation constant of a particular antibody-antigen interaction as is known in the art.

The term “labeled binding protein” as used herein, refers to a protein with a label incorporated that provides for the identification of the binding protein. Preferably, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

The term “antibody conjugate” refers to a binding protein, such as an antibody, chemically linked to a second chemical moiety, such as a therapeutic or cytotoxic agent. The term “agent” is used herein to denote a chemical compound, a mixture of chemical compounds, a biological macromolecule, or an extract made from biological materials. Preferably the therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

The terms “crystal”, and “crystallized” as used herein, refer to an antibody, or antigen binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, Crystallization of Nucleic Acids and Proteins, a Practical Approach, 2nd ea., pp. 20 1-16, Oxford University Press, New York, N.Y., (1999).”

The term “polynucleotide” as referred to herein means a polymeric form of two or more nucleotides, either ribonucleotides or deoxynucleotides or a modified form of either type of nucleotide. The term includes single and double stranded forms of DNA but preferably is double-stranded DNA.

The term “isolated polynucleotide” as used herein shall mean a polynucleotide (e.g., of genomic, cDNA, or synthetic origin, or some combination thereof) that, by virtue of its origin, the “isolated polynucleotide”: is not associated with all or a portion of a polynucleotide with which the “isolated polynucleotide” is found in nature; is operably linked to a polynucleotide that it is not linked to in nature; or does not occur in nature as part of a larger sequence.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest.

The term “expression control sequence” as used herein refers to polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak-consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing, and can also include additional components whose presence is advantageous, for example, leader sequences and fusion partner sequences.

“Transformation”, as defined herein, refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells that transiently express the inserted DNA or RNA for limited periods of time.

The term “recombinant host cell” (or simply “host cell”), as used herein, is intended to refer to a cell into which exogenous DNA has been introduced. It should be understood that such terms are intended to refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. Preferably host cells include prokaryotic and eukaryotic cells selected from any of the Kingdoms of life. Preferred eukaryotic cells include protist, fungal, plant and animal cells. Most preferably host cells include but are not limited to the prokaryotic cell line E. coli; mammalian cell lines CHO, HEK 293 and COS; the insect cell line Sf9; and the fungal cell Saccharomyces cerevisiae.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose.

“Transgenic organism”, as known in the art and as used herein, refers to an organism having cells that contain a transgene, wherein the transgene introduced into the organism (or an ancestor of the organism) expresses a polypeptide not naturally expressed in the organism. A “transgene” is a DNA construct, which is stably and operably integrated into the genome of a cell from which a transgenic organism develops, directing the expression of an encoded gene product in one or more cell types or tissues of the transgenic organism.

The term “regulate” and “modulate” are used interchangeably, and, as used herein, refers to a change or an alteration in the activity of a molecule of interest (e.g., the biological activity of mouse CXCL13). Modulation may be an increase or a decrease in the magnitude of a certain activity or function of the molecule of interest. Exemplary activities and functions of a molecule include, but are not limited to, binding characteristics, enzymatic activity, cell receptor activation, and signal transduction.

Correspondingly, the term “modulator,” as used herein, is a compound capable of changing or altering an activity or function of a molecule of interest (e.g., the biological activity of mouse CXCL13). For example, a modulator may cause an increase or decrease in the magnitude of a certain activity or function of a molecule compared to the magnitude of the activity or function observed in the absence of the modulator. In certain embodiments, a modulator is an inhibitor, which decreases the magnitude of at least one activity or function of a molecule. Exemplary inhibitors include, but are not limited to, proteins, peptides, antibodies, peptibodies, carbohydrates or small organic molecules. Peptibodies are described, e.g., in WO01/83525.

The term “agonist”, as used herein, refers to a modulator that, when contacted with a molecule of interest, causes an increase in the magnitude of a certain activity or function of the molecule compared to the magnitude of the activity or function observed in the absence of the agonist. Particular agonists of interest may include, but are not limited to, mouse CXCL13 polypeptides or polypeptides, nucleic acids, carbohydrates, or any other molecules that bind to mouse CXCL13.

The term “antagonist” or “inhibitor”, as used herein, refer to a modulator that, when contacted with a molecule of interest causes a decrease in the magnitude of a certain activity or function of the molecule compared to the magnitude of the activity or function observed in the absence of the antagonist. Particular antagonists of interest include those that block or modulate the biological or immunological activity of mouse CXCL13. Antagonists and inhibitors of mouse CXCL13 may include, but are not limited to, proteins, nucleic acids, carbohydrates, or any other molecules, which bind to mouse CXCL13.

As used herein, the term “effective amount” refers to the amount of a therapy which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent).

The term “sample”, as used herein, is used in its broadest sense. A “biological sample”, as used herein, includes, but is not limited to, any quantity of a substance from a living thing or formerly living thing. Such living things include, but are not limited to, humans, mice, rats, monkeys, dogs, rabbits and other animals. Such substances include, but are not limited to, blood, serum, urine, synovial fluid, cells, organs, tissues, bone marrow, lymph nodes and spleen.

I. Antibodies that Bind Mouse CXCL13.

One aspect of the present invention provides isolated murine monoclonal antibodies, or antigen-binding portions thereof, that bind to mouse CXCL13 with high affinity, a slow off rate and high neutralizing capacity. A second aspect of the invention provides chimeric antibodies that bind CXCL13. A third aspect of the invention provides CDR grafted antibodies, or antigen-binding portions thereof, that bind CXCL13. Preferably, the antibodies, or portions thereof, are isolated antibodies. Preferably, the antibodies of the invention are neutralizing anti-mouse CXCL13 antibodies.

### A. Method of Making Anti Mouse CXCL13 Antibodies

Antibodies of the present invention may be made by any of a number of techniques known in the art.

### 1. Anti-Mouse CXCL13 Monoclonal Antibodies Using Hybridoma Technology

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. In one embodiment, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention (See Example 1.2). Briefly, mice can be immunized with a mouse CXCL13 antigen. In a preferred embodiment, the mouse CXCL13 antigen is administered with a adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks.

After immunization of an animal with a mouse CXCL13 antigen, antibodies and/or antibody-producing cells may be obtained from the animal. An anti-mouse CXCL13 antibody-containing serum is obtained from the animal by bleeding or sacrificing the animal. The serum may be used as it is obtained from the animal, an immunoglobulin fraction may be obtained from the serum, or the anti-mouse CXCL13 antibodies may be purified from the serum. Serum or immunoglobulins obtained in this manner are polyclonal, thus having a heterogeneous array of properties.

Once an immune response is detected, e.g., antibodies specific for the antigen mouse CXCL13 are detected in the mouse serum, the mouse spleen is harvested and splenocytes isolated. The splenocytes are then fused by well-known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding mouse CXCL13. Ascites fluid, which generally contains high levels of antibodies, can be generated by immunizing mice with positive hybridoma clones.

In another embodiment, antibody-producing immortalized hybridomas may be prepared from the immunized animal. After immunization, the animal is sacrificed and the splenic B cells are fused to immortalized myeloma cells as is well known in the art. See, e.g., Harlow and Lane, supra. In a preferred embodiment, the myeloma cells do not secrete immunoglobulin polypeptides (a non-secretory cell line). After fusion and antibiotic selection, the hybridomas are screened using mouse CXCL13, or a portion thereof; or a cell expressing mouse CXCL13. In a preferred embodiment, the initial screening is performed using an enzyme-linked immunoassay (ELISA) or a radioimmunoassay (RIA), preferably an ELISA. An example of ELISA screening is provided in WO 00/37504, herein incorporated by reference.

Anti-mouse CXCL13 antibody-producing hybridomas are selected, cloned and further screened for desirable characteristics, including robust hybridoma growth, high antibody production and desirable antibody characteristics, as discussed further below. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, the hybridomas are mouse hybridomas, as described above.

In another preferred embodiment, the hybridomas are produced in a non-human, non-mouse species such as rats, sheep, pigs, goats, cattle or horses.

Antibody fragments that recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CHI domain of the heavy chain.

### 2. Anti-Mouse CXCL13 Monoclonal Antibodies Using SLAM

In another aspect of the invention, recombinant antibodies are generated from single, isolated lymphocytes using a procedure referred to in the art as the selected lymphocyte antibody method (SLAM), as described in U.S. Pat. No. 5,627,052, PCT Publication WO 92/02551 and Babcock, J. S. et al. (1996) Proc. Natl. Acad. Sci. USA 93:7843-7848. In this method, single cells secreting antibodies of interest, e.g., lymphocytes derived from any one of the immunized animals described in Section 1, are screened using an antigen-specific hemolytic plaque assay, wherein the antigen mouse CXCL13, a subunit of mouse CXCL13, or a fragment thereof, is coupled to sheep red blood cells using a linker, such as biotin, and used to identify single cells that secrete antibodies with specificity for mouse CXCL13. Following identification of antibody-secreting cells of interest, heavy- and light-chain variable region cDNAs are rescued from the cells by reverse transcriptase-PCR and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example by panning the transfected cells to isolate cells expressing antibodies to mouse CXCL13. The amplified immunoglobulin sequences further can be manipulated in vitro, such as by in vitro affinity maturation methods such as those described in PCT Publication WO 97/29131 and PCT Publication WO 00/56772.

### 3. Anti-Mouse CXCL13 Monoclonal Antibodies Using Recombinant Antibody Libraries

In vitro methods also can be used to make the antibodies of the invention, wherein an antibody library is screened to identify an antibody having the desired binding specificity. Methods for such screening of recombinant antibody libraries are well known in the art and include methods described in, for example, Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, U.S. patent application publication 20030186374, and PCT Publication No. WO 97/29131, the contents of each of which are incorporated herein by reference.

In one aspect, the invention pertains to an isolated antibody, or an antigen-binding portion thereof, that binds mouse CXCL13. Preferably, the antibody is a neutralizing antibody. In various embodiments, the antibody is a recombinant antibody or a monoclonal antibody.

### B. Production of Recombinant Mouse CXCL13 Antibodies

Antibodies of the present invention may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies of the invention in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure are within the scope of the present invention. For example, it may be desirable to transfect a host cell with DNA encoding functional fragments of either the light chain and/or the heavy chain of an antibody of this invention. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than the antigens of interest by crosslinking an antibody of the invention to a second antibody by standard chemical crosslinking methods.

In a preferred system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. Still further the invention provides a method of synthesizing a recombinant antibody of the invention by culturing a host cell of the invention in a suitable culture medium until a recombinant antibody of the invention is synthesized. The method can further comprise isolating the recombinant antibody from the culture medium.

### 1. Anti-CXCL13 Antibodies

Table 2 is a list of amino acid sequences of VH and VL regions of preferred anti-mouse CXCL13 antibodies of the invention.

TABLE 2
List of Amino Acid Sequences of VH and VL regions

| | |
|---|---|
| SEQ | |
| ID | Sequence |

| | | |
|---|---|---|
| No. | Protein region | 123456789012345678901234567890 |

| | | | |
|---|---|---|---|
| 5 | VH | | QVQLQQPGTELVKPGASVKLSCKASGYNFA |
| | XW25-2C4-4H8 | | TYWMHWVKQRPGQGLEWIGNINPSDGGTNF |
| | | | NEKFNNKATLTVDKSSSTAYMQLSSLTSED |
| | | | SAVYYCAKWGGHYVLYWYFDVWGTGTTVTV |
| | | | SS |
| 6 | VH 2C4 CDR-H1 | Residues 31-35 of | TYWMH |
| | | SEQ ID NO.:5 | |
| 7 | VH 2C4 CDR-H2 | Residues 50-66 of | NINPSDGGTNFNEKFNN |
| | | SEQ ID NO.:5 | |
| 8 | VH 2C4 CDR-H3 | Residues 99-111 of | WGGHYVLYWYFDV |
| | | SEQ ID NO.:5 | |
| 9 | VL | | DIVLTQSPATLSVTPGDSVSLSCRASQSIS |
| | XW25-2C4-4H8 | | SNLHWYQQKSHESPRLLITYASQSISGIPS |
| | | | RFSGSGSGTDFTLSINSVESEDVGVYFCQQ |
| | | | SDSWPLTFGAGTKLELKR |
| 10 | VL 2C4 CDR-L1 | Residues 24-34 of | RASQSISSNLH |
| | | SEQ ID NO.:6 | |
| 11 | VL 2C4 CDR-L2 | Residues 50-56 of | YASQSIS |
| | | SEQ ID NO.:6 | |
| 12 | VL 2C4 CDR-L3 | Residues 89-97 of | QQSDSWPLT |
| | | SEQ ID NO.:6 | |
| 13 | VH | | QVQLQQPGTELVKPGTSVKLSCKASGYTFT |
| | XW25-3B5-3F4 | | SYWMHWVKQRPGQGLEWIGNINPSDGGIKY |
| | | | NEKFKSKATLTVDKSSSTAYMELSSLTSED |
| | | | SAVYYCAKWGGHYVLYWYFDVWGTGTTVTV |
| | | | SS |
| 14 | VH 3B5 CDR-H1 | Residues 31-35 of | SYWMH |
| | | SEQ ID NO.:7 | |
| 15 | VH 3B5 CDR-H2 | Residues 50-66 of | NINPSDGGIKYNEKFKS |
| | | SEQ ID NO.:7 | |
| 16 | VH 3B5 CDR-H3 | Residues 99-111 of | WGGHYVLYWYFDV |
| | | SEQ ID NO.:7 | |
| 17 | VL | | DIVLTQSPATLSVTPGDSVSLSCRASQSIS |
| | XW25-3B5-3F4 | | NNLHWYQQKSHESPRLLINYASQSISGIPS |
| | | | RFSGSGSGTDFTLSINSVETEDFGMYFCQQ |
| | | | SNSWPLTFGAGTKLELKR |
| 18 | VL 3B5 CDR-L1 | Residues 24-34 of | RASQSISNNLH |
| | | SEQ ID NO.:8 | |
| 19 | VL 3B5 CDR-L2 | Residues 50-56 of | YASQSIS |
| | | SEQ ID NO.:8 | |
| 20 | VL 3B5 CDR-L3 | Residues 89-97 of | QQSNSWPLT |
| | | SEQ ID NO.8 | |
| 21 | VH | | QVQLQQPGTELVKPGASVKLSCKASGYNFP |
| | XW25-4F2-5D2 | | TYWMHWVKQRPGQGLEWIGNINPSDGGTNF |
| | | | NEKFNNKATLTVDKSSSTAYMQLSSLTSED |
| | | | SAVYYCAKWGGHYVLYWYFDVWGTGTTVTV |
| | | | SS |
| 22 | VH 4F2 CDR-H1 | Residues 31-35 of | TYWMH |
| | | SEQ ID NO.:9 | |
| 23 | VH 4F2 CDR-H2 | Residues 50-66 of | NINPSDGGTNFNEKFNN |
| | | SEQ ID NO.:9 | |
| 24 | VH 4F2 CDR-H3 | Residues 99-111 of | WGGHYVLYWYFDV |
| | | SEQ ID NO.:9 | |
| 25 | VL | | DIVLTQSPATLSVTPGDSVSLSCRASQSIS |
| | XW25-4F2-5D2 | | NNLHWYQQKSHESPRLLITYASQSISGIPS |
| | | | RFSGSGSGTDFTLSINSVESEDVGVYFCQQ |
| | | | SDSWPLTFGAGTKLELKR |
| 26 | VL 4F2 CDR-L1 | Residues 24-34 of | RASQSISNNLH |
| | | SEQ ID NO.:10 | |
| 27 | VL 4F2 CDR-L2 | Residues 50-56 of | YASQSIS |
| | | SEQ ID NO.:10 | |
| 28 | VL 4F2 CDR-L3 | Residues 89-97 of | QQSDSWPLT |
| | | SEQ ID NO.:10 | |
| 29 | VH | | EVQVQQSGAELVRPGSSVKMSCKTSGYTFT |
| | XW25-5D11- | | NYGIIWVKQRPGQGLEWIGYIYIGKGYTEY |
| | 4F3-3D11 | | NEKFKGKATLTSDTSSSTVYMQLSSLTSED |
| | | | SAIYFCARGAHYYGNAMDYWDQGTSVTVSS |
| 30 | VH 5D11 CDR-H1 | Residues 31-35 of | NYGII |
| | | SEQ ID NO.:11 | |
| 31 | VH 5D11 CDR-H2 | Residues 50-66 of | YIYIGKGYTEYNEKFKG |
| | | SEQ ID NO.:11 | |
| 32 | VH 5D11 CDR-H3 | Residues 99-109 of | GAHYYGNAMDY |
| | | SEQ ID NO.:11 | |
| 33 | VL | | DIQMTQTTSSLSASLGDRVTISCRSSQDIS |
| | XW25-5D11- | | NFLNWYQQRPDGTVKLLIYYTSRLHSGVPS |
| | 4F3-3D11 | | RFSGSGSGTDYSLTISNLEQEDIATYFCQQ |
| | | | GDTLPWTFGGGTKLEIKR |
| 34 | VL 5D11 CDR-L1 | Residues 24-34 of | RSSQDISNFLN |
| | | SEQ ID NO.:12 | |
| 35 | VL 5D11 CDR-L2 | Residues 50-56 of | YTSRLHS |
| | | SEQ ID NO.:12 | |
| 36 | VL 5D11 CDR-L3 | Residues 89-97 of | QQGDTLPWT |
| | | SEQ ID NO.:12 | |
| 37 | VH | | QVQLQQPGTELVKPGASVKLSCKASGYNFA |
| | XW25-6A2-2F10 | | TYWVHWVKQRPGQGLEWIGNINPSDGGTSY |
| | | | NEKFSKKATLTVDKSSSTAYMELSSLTSED |
| | | | SAVYYCAKWGGHYVLYWSFDVWGTGTTVTV |
| | | | SS |
| 38 | VH 6A2 CDR-H1 | Residues 31-35 of | TYWVH |
| | | SEQ ID NO.:13 | |
| 39 | VH 6A2 CDR-H2 | Residues 50-66 of | NINPSDGGTSYNEKFSK |
| | | SEQ ID NO.:13 | |
| 40 | VH 6A2 CDR-H3 | Residues 99-111 of | WGGHYVLYWSFDV |
| | | SEQ ID NO.:13 | |
| 41 | VL | | DIVLTQSPATLSVTPGDSVSLSCRASQSIS |
| | XW25-6A2-2F10 | | NNLHWYQQRSHESPRLIITHVSQSISGIPS |
| | | | RFSGSGSGTDFIFSINSVESEDFGVYFCQQ |
| | | | SNSWPLTFGAGTKLELKR |
| 42 | VL 6A2 CDR-L1 | Residues 24-34 of | RASQSISNNLH |
| | | SEQ ID NO.:14 | |
| 43 | VL 6A2 CDR-L2 | Residues 50-56 of | HVSQSIS |
| | | SEQ ID NO.:14 | |
| 44 | VL 6A2 CDR-L3 | Residues 89-97 of | QQSNSWPLT |
| | | SEQ ID NO.:14 | |
| 45 | VH | | QVQLQQPGTELVKPGASVKLSCKASGYNFA |
| | XW25-7C7-5E5 | | TYWMHWVKQRPGQGLEWIGNINPSDGGTNF |
| | | | NEKFNNKATLTVDKSSSTAYMHLSSLTSED |
| | | | SAVYYCAKWGGHYVLYWYFDVWGTGTTVTV |
| | | | SS |
| 46 | VH 7C7 CDR-H1 | Residues 31-35 of | TYWMH |
| | | SEQ ID NO.:15 | |
| 47 | VH 7C7 CDR-H2 | Residues 50-66 of | NINPSDGGTNFNEKFNN |
| | | SEQ ID NO.:15 | |
| 48 | VH 7C7 CDR-H3 | Residues 99-111 of | WGGHYVLYWYFDV |
| | | SEQ ID NO.:15 | |
| 49 | VL | | DIVLTQSPATLSVTPGDSVSLSCRASQNIN |
| | XW25-7C7-5E5 | | NNLHWYQQKSHESPRLLITYTSQSISGIPS |
| | | | RFSGSGSGTDFTLSINSVESEDVGVYFCQQ |
| | | | SDSWPLTFGAGTKLELKR |
| 50 | VL 7C7 CDR-L1 | Residues 24-34 of | RASQNINNNLH |
| | | SEQ ID NO.:16 | |
| 51 | VL 7C7 CDR-L2 | Residues 50-56 of | YTSQSIS |
| | | SEQ ID NO.:16 | |
| 52 | VL 7C7 CDR-L3 | Residues 89-97 of | QQSDSWPLT |
| | | SEQ ID NO.:16 | |
| 53 | VH | | QVQLQQPGTELVKPGASVKLSCKASGYNFA |
| | XW25-8H6-4F9 | | TYWMHWVKQRPGQGLEWIGNINPSDGGTNY |
| | | | NEKFNNKATLTVDKSSSTAYMQLSSLTSED |
| | | | SAVYYCAKWGGHYVLYWYFDVWGTGTTVTV |
| | | | SS |
| 54 | VH 8H6 CDR-H1 | Residues 31-35 of | TYWMH |
| | | SEQ ID NO.:17 | |
| 55 | VH 8H6 CDR-H2 | Residues 50-66 of | NINPSDGGTNYNEKFNN |
| | | SEQ ID NO.:17 | |
| 56 | VH 8H6 CDR-H3 | Residues 99-111 of | WGGHYVLYWYFDV |
| | | SEQ ID NO.:17 | |
| 57 | VL | | DIVLTQSPATLSVTPGDSVSLSCRASQSIN |
| | XW25-8H6-4F9 | | NNLHWYQQESHESPRLLITYASQSISGIPS |
| | | | RFSGSGSGTDFTLSINSVESEDFGVYFCQQ |
| | | | SNSWPLTFGAGTKLELKR |
| 58 | VL 8H6 CDR-L1 | Residues 24-34 of | RASQSINNNLH |
| | | SEQ ID NO.:18 | |
| 59 | VL 8H6 CDR-L2 | Residues 50-56 of | YASQSIS |
| | | SEQ ID NO.:18 | |
| 60 | VL 8H6 CDR-L3 | Residues 89-97 of | QQSNSWPLT |
| | | SEQ ID NO.:18 | |
| 61 | VH | | QVQLQQPGTELVKPGASVKLSCKASGYNFA |
| | XW25-9C10- | | TYWMHWVKQRPGQGLEWIGNINPSDGGTNF |
| | 4B11 | | NEKFNSKATLTVDKSSSTAYMQLSSLTSED |
| | | | SAVYYCAKWGGHYVLYWYFDVWGTGTTVTV |
| | | | SS |
| 62 | VH 9C10 CDR-H1 | Residues 31-35 of | TYWMH |
| | | SEQ ID NO.:19 | |
| 63 | VH 9C10 CDR-H2 | Residues 50-66 of | NINPSDGGTNFNEKFNS |
| | | SEQ ID NO.:19 | |
| 64 | VH 9C10 CDR-H3 | Residues 99-111 of | WGGHYVLYWYFDV |
| | | SEQ ID NO.:19 | |
| 65 | VL | | DIVLTQSPATLSVTPGDSVSLSCRASQSIS |
| | XW25-9C10- | | SNLHWYQQKSHESPRLLITYASQSISGIPS |
| | 4B11 | | RFSGSGSGTDFTLSINSVESEDVGVYFCQQ |
| | | | SDSWPLTFGAGTKLELKR |
| 66 | VL 9C10 CDR-L1 | Residues 24-34 of | RASQSISSNLH |
| | | SEQ ID NO.:20 | |
| 67 | VL 9C10 CDR-L2 | Residues 50-56 of | YASQSIS |
| | | SEQ ID NO.:20 | |
| 68 | VL 9C10 CDR-L3 | Residues 89-97 of | QQSDSWPLT |
| | | SEQ ID NO.:20 | |

The foregoing isolated anti-mouse CXCL13 antibody CDR sequences establish a novel family of mouse CXCL13 binding proteins, isolated in accordance with this invention, and comprising polypeptides that include the CDR sequences listed. To generate and to select CDR's of the invention having preferred mouse CXCL13 binding and/or neutralizing activity with respect to mouse CXCL13, standard methods known in the art for generating binding proteins of the present invention and assessing the mouse CXCL13 and or mouse CXCL13 binding and/or neutralizing characteristics of those binding protein may be used, including but not limited to those specifically described herein.

### C. Production of Antibodies and Antibody-Producing Cell Lines

Preferrably, anti-CXCL13 antibodies of the present invention, exhibit a high capacity to reduce or to neutralize mouse CXCL13 activity, e.g., as assessed by any one of several in vitro and in vivo assays known in the art (e.g., see Example 1.1.C). For example, these antibodies neutralize CXCL13-induced chemotaxis of or calcium flux in cells containing the CXCR5 receptor with IC₅₀ values in the range of at least about 10⁻⁸ M, about 10⁻⁹ M, or about 10⁻¹⁰ M. Preferrably, anti-mouse CXCL13 antibodies of the present invention, also exhibit a high capacity to reduce or to neutralize mouse CXCL13 activity

In preferred embodiments, the isolated antibody, or antigen-binding portion thereof, binds mouse CXCL13, wherein the antibody, or antigen-binding portion thereof, dissociates from mouse CXCL13 with a k_{off} rate constant of about 0.1 s⁻¹ or less, as determined by surface plasmon resonance, or which inhibits mouse CXCL13 activity with an IC₅₀ of about 1×10⁻⁶M or less. Alternatively, the antibody, or an antigen-binding portion thereof, may dissociate from mouse CXCL13 with a k_{off} rate constant of about 1×10⁻² s⁻¹ or less, as determined by surface plasmon resonance, or may inhibit mouse CXCL13 activity with an IC₅₀ of about 1×10⁻⁷M or less. Alternatively, the antibody, or an antigen-binding portion thereof, may dissociate from mouse CXCL13 with a k_{off} rate constant of about 1×10⁻³ s⁻¹ or less, as determined by surface plasmon resonance, or may inhibit mouse CXCL13 with an IC₅₀ of about 1×10⁻⁸M or less. Alternatively, the antibody, or an antigen-binding portion thereof, may dissociate from mouse CXCL13 with a k_{off} rate constant of about 1×10⁻⁴ s⁻¹ or less, as determined by surface plasmon resonance, or may inhibit mouse CXCL13 activity with an IC₅₀ of about 1×10⁻⁹M or less. Alternatively, the antibody, or an antigen-binding portion thereof, may dissociate from mouse CXCL13 with a k_{off} rate constant of about 1×10⁻⁵ s⁻¹ or less, as determined by surface plasmon resonance, or may mouse CXCL13 activity with an IC₅₀ of about 1×10⁻¹⁰ M or less. Alternatively, the antibody, or an antigen-binding portion thereof, may dissociate from mouse CXCL13 with a k_{off} rate constant of about 1×10⁻⁶ s⁻¹ or less, as determined by surface plasmon resonance, or may inhibit mouse CXCL13 activity with an IC₅₀ of about 1×10⁻¹¹M or less.

In certain embodiments, the antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. Preferably, the antibody comprises a kappa light chain constant region. Alternatively, the antibody portion can be, for example, a Fab fragment or a single chain Fv fragment.

Replacements of amino acid residues in the Fc portion to alter antibody effector function are known in the art (Winter, et al. U.S. Pat. Nos. 5,648,260; 5,624,821). The Fc portion of an antibody mediates several important effector functions e.g. cytokine induction, ADCC, phagocytosis, complement dependent cytotoxicity (CDC) and half-life/clearance rate of antibody and antigen-antibody complexes. In some cases these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to FcγR5 and complement C1q, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered.

One embodiment provides a labeled binding protein wherein an antibody or antibody portion of the invention is derivatized or linked to another functional molecule (e.g., another peptide or protein). For example, a labeled binding protein of the invention can be derived by functionally linking an antibody or antibody portion of the invention (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

Useful detectable agents with which an antibody or antibody portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

Another embodiment of the invention provides a crystallized binding protein. Preferably the invention relates to crystals of whole anti-MOUSE CXCL13 antibodies and fragments thereof as disclosed herein, and formulations and compositions comprising such crystals. In one embodiment the crystallized binding protein has a greater half-life in vivo than the soluble counterpart of the binding protein. In another embodiment the binding protein retains biological activity after crystallization.

Crystallized binding protein of the invention may be produced according methods known in the art and as disclosed in WO 02072636, incorporated herein by reference.

Another embodiment of the invention provides a glycosylated binding protein wherein the antibody or antigen-binding portion thereof comprises one or more carbohydrate residues. Nascent in vivo protein production may undergo further processing, known as post-translational modification. In particular, sugar (glycosyl) residues may be added enzymatically, a process known as glycosylation. The resulting proteins bearing covalently linked oligosaccharide side chains are known as glycosylated proteins or glycoproteins. Protein glycosylation depends on the amino acid sequence of the protein of interest, as well as the host cell in which the protein is expressed. Different organisms may produce different glycosylation enzymes (e.g., glycosyltransferases and glycosidases), and have different substrates (nucleotide sugars) available. Due to such factors, protein glycosylation pattern, and composition of glycosyl residues, may differ depending on the host system in which the particular protein is expressed. Glycosyl residues useful in the invention may include, but are not limited to, glucose, galactose, mannose, fucose, n-acetylglucosamine and sialic acid. Preferably the glycosylated binding protein comprises glycosyl residues such that the glycosylation pattern is human.

It is known to those skilled in the art that differing protein glycosylation may result in differing protein characteristics. For instance, the efficacy of a therapeutic protein produced in a microorganism host, such as yeast, and glycosylated utilizing the yeast endogenous pathway may be reduced compared to that of the same protein expressed in a mammalian cell, such as a CHO cell line. Such glycoproteins may also be immunogenic in humans and show reduced half-life in vivo after administration. Specific receptors in humans and other animals may recognize specific glycosyl residues and promote the rapid clearance of the protein from the bloodstream. Other adverse effects may include changes in protein folding, solubility, susceptibility to proteases, trafficking, transport, compartmentalization, secretion, recognition by other proteins or factors, antigenicity, or allergenicity. Accordingly, a practitioner may prefer a therapeutic protein with a specific composition and pattern of glycosylation, for example glycosylation composition and pattern identical, or at least similar, to that produced in human cells or in the species-specific cells of the intended subject animal.

Expressing glycosylated proteins different from that of a host cell may be achieved by genetically modifying the host cell to express heterologous glycosylation enzymes. Using techniques known in the art a practitioner may generate antibodies or antigen-binding portions thereof exhibiting human protein glycosylation. For example, yeast strains have been genetically modified to express non-naturally occurring glycosylation enzymes such that glycosylated proteins (glycoproteins) produced in these yeast strains exhibit protein glycosylation identical to that of animal cells, especially human cells (U.S. patent applications 20040018590 and 20020137134).

Further, it will be appreciated by one skilled in the art that a protein of interest may be expressed using a library of host cells genetically engineered to express various glycosylation enzymes, such that member host cells of the library produce the protein of interest with variant glycosylation patterns. A practitioner may then select and isolate the protein of interest with particular novel glycosylation patterns. Preferably, the protein having a particularly selected novel glycosylation pattern exhibits improved or altered biological properties.

### D. Uses of Anti-Mouse CXCL13 Antibodies

Given their ability to bind to mouse CXCL13, the anti-mouse CXCL13 antibodies, or portions thereof, of the invention can be used to detect mouse CXCL13 (e.g., in a biological sample, such as serum or plasma), using a conventional immunoassay, such as an enzyme linked immunosorbent assays (ELISA), an radioimmunoassay (RIA) or tissue immunohistochemistry. The invention provides a method for detecting mouse CXCL13 in a biological sample comprising contacting a biological sample with an antibody, or antibody portion, of the invention and detecting either the antibody (or antibody portion) bound to mouse CXCL13 or unbound antibody (or antibody portion), to thereby detect mouse CXCL13 in the biological sample. The antibody is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ³H ¹⁴C ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm.

Alternative to labeling the antibody, mouse CXCL13 can be assayed in biological fluids by a competition immunoassay utilizing recombinant mouse CXCL13 standards labeled with a detectable substance and an unlabeled anti-mouse CXCL13 antibody. In this assay, the biological sample, the labeled recombinant mouse CXCL13 standards and the anti-mouse CXCL13 antibody are combined and the amount of labeled recombinant mouse CXCL13 standard bound to the unlabeled antibody is determined. The amount of mouse CXCL13 in the biological sample is inversely proportional to the amount of labeled recombinant mouse CXCL13 standard bound to the anti-mouse CXCL13 antibody. Similarly, mouse CXCL13 can also be assayed in biological fluids by a competition immunoassay utilizing recombinant mouse CXCL13 standards labeled with a detectable substance and an unlabeled anti-mouse CXCL13 antibody.

The antibodies and antibody portions of the invention preferably are capable of neutralizing mouse CXCL13 activity both in vitro and in vivo. Accordingly, such antibodies and antibody portions of the invention can be used to inhibit mouse CXCL13 activity, e.g., in a cell culture containing mouse CXCL13, in an animan having mouse CXCL13 with which an antibody of the invention cross-reacts. In one embodiment, the invention provides a method for inhibiting mouse CXCL13 activity comprising contacting mouse CXCL13 with an antibody or antibody portion of the invention such that mouse CXCL13 activity is inhibited. For example, in a cell culture containing, or suspected of containing mouse CXCL13, an antibody or antibody portion of the invention can be added to the culture medium to inhibit mouse CXCL13 activity in the culture.

In another embodiment, the invention provides a method for reducing mouse CXCL13 activity in an animal, advantageously from an animal suffering from a disease or disorder in which mouse CXCL13 activity is detrimental. The invention provides methods for reducing mouse CXCL13 activity in an animal suffering from such a disease or disorder, which method comprises administering to the animal an antibody or antibody portion of the invention such that mouse CXCL13 activity in the animal is reduced. Alternatively, the animal can be a mammal expressing a mouse CXCL13 to which an antibody of the invention is capable of binding. Still further the animal can be a mammal into which mouse CXCL13 has been introduced (e.g., by administration of mouse CXCL13 or by expression of a mouse CXCL13 transgene). An antibody of the invention can be administered to an animal for therapeutic purposes. Moreover, an antibody of the invention can be administered to a non-human mammal expressing a mouse CXCL13 with which the antibody is capable of binding for veterinary purposes or as an animal model of human disease. Regarding the latter, such animal models may be useful for evaluating the therapeutic efficacy of antibodies of the invention (e.g., testing of dosages and time courses of administration).

As used herein, the term “a disorder in which mouse CXCL13 activity is detrimental” is intended to include diseases and other disorders in which the presence of mouse CXCL13 in an animal suffering from the disorder has been shown to be or is suspected of being either responsible for the pathophysiology of the disorder or a factor that contributes to a worsening of the disorder. Accordingly, a disorder in which mouse CXCL13 activity is detrimental is a disorder in which reduction of mouse CXCL13 activity is expected to alleviate the symptoms and/or progression of the disorder. Such disorders may be evidenced, for example, by an increase in the concentration of mouse CXCL13 in a biological fluid of an animal suffering from the disorder (e.g., an increase in the concentration of mouse CXCL13 in serum, plasma, synovial fluid, etc. of the animal), which can be detected, for example, using an anti-mouse CXCL13 antibody as described above. Non-limiting examples of disorders include, but are not limited to, rheumatoid arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjörgren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma). The antibodies, and antibody portions of the invention can be used to treat animals suffering from autoimmune diseases, in particular those associated with inflammation, including, rheumatoid spondylitis, allergy, autoimmune diabetes, autoimmune uveitis, and lupus related disorders such as Drug-induced lupus; Cutaneous lupus (malar rash, photosensitivity); Discoid lupus (discoid rash, photosensitivity); lupus with Antiphospholipid Antibody Syndrome; lupus nephritis including (Minimal change, Mesangial, FSGN, PGN, Membranous (proteinuria, urinary casts); non-nephritic lupus with hematological manifestations (leukopenia, lymphopenia, thrombocytopenia, hemolytic anemia, vasculitis); non-nephritic lupus with Cardio-pulmonary manifestations (Pericarditis, Pleuritis); CNS lupus (seizure, psychosis, organic brain syndrome, visual disturbance, cranial nerve disorder, lupus headache, cerebrovascular accidents, lupus fever, vasculitis); mild lupus (fatigue, skin rashes, arthritis, athrialgia, myalgia, mouth sores, hair loss (alopecia); moderate lupus (serositis, severe rash, nephritis); severe lupus (diffuse proliferative glomerulonephritis, vasculitic ulcers, CNS inflammation); steroid refractory Chemotherapy (ie, cyclophosphamide) refractory; Adult lupus; Childhood and adolescence lupus; other lupus related Hematological disorders such as leukopenia, lymphopenia, thrombocytopenia, hemolytic anemia, vasculitis, adenopathy, lupus related Cardio-pulmonary disorders such as Pericarditis, Pleuritis; lupus related joint disorder such as arthritis; and lupus related Immunologic disorders such as Anti-DNA, Anti-Ro, Anti-Sm. Anti-ANA, high gamma globulin, low complement, myositis, and mucosal ulcers.

A “therapeutically effective amount” refers to an amount effective, at dosages and for periods of time necessary, to achieve the desired therapeutic result. A therapeutically effective amount of the antibody or antibody portion may be determined by a person skilled in the art and may vary according to factors such as the disease state, age, sex, and weight of the individual, and the ability of the antibody or antibody portion to elicit a desired response in the individual. A therapeutically effective amount is also one in which any toxic or detrimental effects of the antibody, or antibody portion, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of an antibody or antibody portion of the invention is 0.1-20 mg/kg, more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

It will be readily apparent to those skilled in the art that other suitable modifications and adaptations of the methods of the invention described herein are obvious and may be made using suitable equivalents without departing from the scope of the invention or the embodiments disclosed herein. Having now described the present invention in detail, the same will be more clearly understood by reference to the following examples, which are included for purposes of illustration only and are not intended to be limiting of the invention.

### EXEMPLIFICATION

### Example 1

### Generation and Isolation of Anti Mouse CXCL13 Monoclonal Antibodies

### Example 1.1

### Assays to Identify Anti Mouse CXCL13 Antibodies

Throughout Example 1 the following assays were used to identify and characterize anti-mouse CXCL13 antibodies unless otherwise stated.

### Example 1.1.A

### ELISA

Enzyme Linked Immunosorbent Assays to screen for antibodies that bind mouse CXCL13 were performed as follows.

### Example 1.1.A.1

### ELISA to Detect Binding of Anti Mouse CXCL13 Antibodies to Mouse CXCL13

ELISA plates (Corning Costar, Acton, Mass.) were coated with 50 μL/well of 5 μg/ml goat anti-mouse IgG Fc specific (Pierce # 31170, Rockford, Ill.) in Phosphate Buffered Saline (PBS) overnight at 4 degrees Celsius. Plates were washed once with PBS containing 0.05% Tween-20. Plates were blocked by addition of 200 μL/well blocking solution diluted to 2% in PBS (BioRad #170-6404, Hercules, Calif.) for 1 hour at room temperature. Plates were washed once after blocking with PBS containing 0.05% Tween-20.

Fifty microliters per well of mouse sera or hybridoma supernatants diluted in PBS containing 0.1% Bovine Serum Albumin (BSA) (Sigma, St. Louis, Mo.) was added to the ELISA plate prepared as described above and incubated for 1 hour at room temperature. Wells were washed three times with PBS containing 0.05% Tween-20. Fifty microliters of biotinylated recombinant purified mouse CXCL13 diluted to 100 ng/mL in PBS containing 0.1% BSA was added to each well and incubated for 1 hour at room temperature. Plates were washed 3 times with PBS containing 0.05% Tween-20. Streptavidin HRP (Pierce # 21126, Rockland, Ill.) was diluted 1:20000 in PBS containing 0.1% BSA; 50 μL/well was added and the plates incubated for 1 hour at room temperature. Plates were washed 3 times with PBS containing 0.05% Tween-20. Fifty microliters of TMB solution (Sigma # T0440, St. Louis, Mo.) was added to each well and incubated for 10 minutes at room temperature. The reaction was stopped by addition of 1N sulphuric acid. Plates were read spectrophotmetrically at a wavelength of 450 nm.

### Example 1.1.B

### Affinity Determinations Using Biacore Technology

The purpose of this study is to evaluate the binding affinities of the purified hybridoma candidates by determining the association rate (kₐ), dissociation rate (k_{d}), and overall binding affinity (K_{d}) of Mouse Anti Mouse CXCL13 Mabs for mouse CXCL13 using a Biacore instrument.

### Example 1.1.B.(a) Materials and Methods

Biacore assay (Biacore, Inc, Piscataway, N.J.) determines the affinity of antibodies with kinetic measurements of association rate (on-rate), dissociation rate (off-rate) constants. Binding of antibodies to recombinant purified mouse CXCL13 were determined by surface plasmon resonance-based measurements with a Biacore® 2000 and 3000 instruments (Biacore® AB, Uppsala, Sweden) using running HBS-EP (10 mM HEPES pH 7.4, 150 mM NaCl, 3 mM EDTA, and 0.015% surfactant P20) at 25° C. Experimental data from all kinetic rate determinations of the antibody-antigen reaction were analyzed using Biaevaluation software version 4.0.1.

All chemicals were obtained from Biacore® AB (Uppsala, Sweden) or otherwise from a different source as described in the text: CM4 biosensor chip: Biacore AB, catalog no BR-10005-39; HBS-EP with 0.01% P20: Biacore AB, catalog no BR-1001-88; Surfactant P20 (10% solution) Biacore AB, catalog no BR-1000-54; 10 mM sodium acetate, pH 4.5: Biacore AB, catalog no BR-1003-50; Amine coupling kits: Biacore AB, catalog no BR-1000-50; 10 mM Glycine pH 1.5: Biacore AB, catalog no BR-1003-54; Goat anti-murine IgG (Fc): Pierce Biotechnology Inc, catalog no 31170, Rockford, Ill.; Goat IgG-UNLB: Southern Biotechnology, catalog no 0109-01, lot K2104 NA66, Birmingham, Ala.; Recombinant murine CXCL13 from R&D, catalog no 470BC/CF, batch no BPPO₄₅₀₇₁, Minneapolis, Minn.

Approximately 2500-5000 RU of goat anti-mouse IgG, (Fcγ), fragment specific polyclonal antibody (Pierce Biotechnology Inc, Rockford, Ill.) and goat IgG (Southern Biotechnology, Birmingham, Ala.) used as reference surface, diluted in 10 mM sodium acetate (pH 4.5), were directly immobilized across a CM4 research grade biosensor chip using a standard amine coupling kit according to manufacturer's instructions and procedures at 25 μg/ml. Unreacted moieties on the biosensor surface were blocked with ethanolamine. Modified carboxymethyl dextran surface with goat anti-mouse IgG Fc in flow cell 2 and 4 was used as a reaction surface. Modified carboxymethyl dextran surface with goat IgG in flow cell 1 and 3 was used as the reference surface.

### Example 1.1.B.(b) Binding of mouse CXCL13 antigen to captured Mouse Anti Mouse Monoclonal CXCL13 antibodies

Mouse Anti Mouse CXCL13 antibodies to be captured as a ligand on the carboxymethyl dextran matrix were diluted in HBS-EP with 0.015% P20 at a concentration of 5-μg/mL. 50-100 μL aliquots were injected over the covalently coupled goat anti-mouse IgG Fc polyclonal antibody biosensor matrices at a flow rate of 25-50 μL/min. The net difference in the baseline signal and the signal after the completion of antibody injection was taken to represent the amount of bound Mouse Anti Mouse CXL13 antibody. Biosensor matrices were typically regenerated with subsequent 25 μL injections of 10 mM Glycine (pH 1.5) before the injection of the next sample. Glycine treatment disrupts the association of non-covalently bound proteins thus Anti CXCL13 antibody-mouse CXCL13 complex dissociates and washes off. The anti-mouse IgG antibody immobilized surfaces were completely regenerated. Aliquots of mouse CXCL13 antigen were injected at a flow rate of 25-50 μL/min over Anti CXCL13 purified monoclonal antibodies captured on the biosensor chips. The concentrations of mouse CXCL13 was ranging between 0.78-50 nM. HBS-EP with 0.015% P20 was flowed through each flow cell in between various cycles of antigens and also immediately before and after the end of kinetic analysis. The signal stabilized 120-180 seconds after the completion of the injection cycle. The net difference in signal between the baseline and the point corresponding to approximately 30 seconds after the completion of antigen injections was taken to represent the amount of antigen bound. The response was measured in resonance units (RU) representing the mass of bound CXCL13 antigen. CXCL13 antigen was also simultaneously injected over the goat anti mouse IgG Fc or goat IgG reference and reaction CM4 surface to record any nonspecific binding background. By using a reference and reaction surface, the reference surface data can be automatically subtracted from the reaction surface data in order to eliminate the majority of the refractive index change and injection noise.

### Example 1.1.B.(c)

### Determination of Association, Dissociation and Apparent Equilibrium Dissociation Rate Constants by Biacore

For kinetic analysis, rate equations derived from the 1:1 Langmuir binding model were fitted simultaneously to association and dissociation phases of all antigen concentration injections (using global fit analysis) with the use of Biaevaluation 4.0.1 software. The association and dissociation rate constants, kon (unit M-1 s-1) and koff (unit s-1) were determined under a continuous flow rate of 25-50 μl/min. Rate constants were derived by making kinetic binding measurements at eight to ten different antigen concentrations ranging from 0.78-50 nM.

The equilibrium dissociation constant (unit M) of the reaction between mouse antibodies and recombinant purified mouse CXCL13 was then calculated from the kinetic rate constants by the following formula: KD=koff/kon. Binding is recorded as a function of time and kinetic rate constants are calculated. In this assay, on-rates as fast as 106 M-1 s-1 and off-rates as slow as 10-6 s-1 can be measured.

The binding affinities of the monoclonal antibodies to recombinant purified mouse CXCL13 were determined using surface plasmon resonance (Biacore®) measurement as described in Example 1.1.B. Table 3 shows the affinity of the monoclonal antibodies described above for mouse CXCL13.

TABLE 3
Affinity of Mouse Anti Mouse CXCL13 Monoclonal
Antibodies for Mouse CXCL13

| Captured | | On-rate | Off-rate | |
|---|---|---|---|---|
| Anti-Murine | | kₒₙ | k_{off} | K_{d} |
| CXCL13 Mab | Isotype | (1/M*s) | (1/s) | (pM) |
| 2C4 | IgG₁ | 2.7 × 10⁶ | 3.6 × 10⁻⁵ | 13 |
| 6A2 | IgG₁ | 1.6 × 10⁶ | 2.7 × 10⁻⁶ | 2 |
| 5D11 | IgG_{2b} | 1.0 × 10⁶ | 3.2 × 10⁻⁴ | 3 |

### Example 1.1.C

### Functional Activity of Anti Mouse CXCL13 Antibodies

To examine the functional activity of the anti-mouse CXCL13 antibodies of the invention, the antibodies were used in the following assays that measure the ability of an antibody to inhibit CXCL13 activity.

### Example 1.1.C1 Calcium Flux Assay in a FLIPR Format to Demonstrate Inhibition of Mouse CXCL13 Activity

The FLIPR assay (Molecular Devices Corp. 1311 Orleans Drive, Sunnyvale, Calif. 94089-1136, USA) determines the ability of antibodies to block the binding of a specific ligand and subsequent signaling through a specific G protein coupled receptor based upon measurement of resultant intracellular calcium flux. Binding of antibodies to recombinant purified mouse CXCL13 were assessed for their ability to inhibit the pre-defined level of receptor signaling of HEK293/Gα16/murine CXCR5 cells when stimulated with a pre-determined concentration of purified murine CXCL13. Receptor signaling was determined by fluorescent measurement of resultant total intracellular calcium mobilization. Experimental antibodies were tested in comparison to a commercially available anti-murine CXCL13 neutralizing antibody (R&D Systems; MAB 470).

HEK293 cells engineered to co-express the murine CXCR5 receptor and murine Gα16 protein were propagated in DMEM medium (Invitrogen) supplemented with 10% heat inactivated Fetal Bovine Serum (SeraCare Life Sciences, Inc.), 1% Penicillin—Streptomycin (Invitrogen), 1% L-glutamine (Invitrogen), 1% non-essential amino acids (Invitrogen), 1% Sodium Pyruvate (Invitrogen), 0.75 mg/ml Geneticin® (Invitrogen), and 0.3 mg/ml Hygromycin (Invitrogen). The cells were plated at a density of 20,000 cells/well at least 12 hours prior to the assay in poly-d lysine coated black wall, clear bottom plates (Corning, Inc.). Recombinant murine CXCL13 (R&D systems) was used as the control agonist for these experiments.

The calcium indicator dye Fluo-4, AM Ester (Invitrogen) was used for fluorescence visualization of intracellular calcium transients. Cells were dye loaded with a 50 ug aliquot of Fluo-4 AM was solublized in 25 ul DMSO (Sigma-Aldrich). Next 50 ul of Pluronic F-127 (Calbiochem) was added and mixed. The total 75 ul was then added to 20 mls of FLIPR buffer (1×HBSS (Invitrogen), 25 mM HEPES (Invitrogen), 0.1% BSA (Invitrogen), and 2.5 mM Probenicid (Sigma-Aldrich)) resulting in a 2.5 uM dye solution. The plating medium was removed and 60 ul/well dye solution was added. The cells+dye solution was incubated at room temperature for 1 hour. The dye solution was removed and replaced with 60 ul FLIPR buffer and incubated at room temperature for an additional 10 minutes.

Antibodies, and chemokine were suspended in FLIPR buffer. A serial dilution of anti-murine CXCL13 Antibodies (ranging from 30 ug/ml-0.03 ug/ml) was incubated with a fixed concentration of murine CXCL 13 for 30 minutes at room temperature. The murine CXCL 13 concentration used was defined as the EC70 of the FLIPR dose response of the HEK Gα16/murineCXCR5 to murine CXCL13. Typically the EC70 of the FLIPR dose response of the HEK Gα16/murineCXCR5 to murine CXCL13 value was 100 nM.

Using the FLIPR instrument, 20 ul of pre-incubated chemokine and antibody in FLIPR buffer were added to the dye loaded cells in 60 ul of FLIPR buffer (total volume 80 ul). Measurements of specific well fluorescence were taken at 1 second intervals for the first minute then 6 second intervals 2 minutes. The total read time was 3 minutes. The maximal fluorescence value for each well was exported as the raw data for the assay. Raw data was analyzed using GraphPad Prism® 4 software (GraphPad Software, Inc.). IC50 values for the chemotaxis neutralizing antibodies were calculated using the non-linear regression and Kb values calculated using a modified Cheng-Prusoff equation (Leff and Dougall, Trends Pharmacol. Sci. 14 (4), 110-2 (1993))

### Example 1.1.C 2 Chemotaxis Assay to Demonstrate Inhibition of Mouse CXCL13 Activity

The chemotaxis assay quantifies the migration of cells toward a chemoattractant. Monoclonal antibodies to recombinant mouse CXCL13 were assessed for their ability to inhibit chemotaxis of recombinant BA/F3 murine CXCR5 cells towards purified mouse CXCL13. Experimental antibodies were tested in comparison to a commercially available anti-murine CXCL13 neutralizing antibody (R&D Systems; MAB 470).

BA/F3 cells (ATCC) engineered to express the murine CXCR5 receptor were propagated in RPMI 1640 medium (Invitrogen) supplemented with 10% heat inactivated Fetal Bovine Serum (SeraCare Life Sciences, Inc.), 1% Penicillin-Streptomycin (Invitrogen), 1% L-glutamine (Invitrogen), 1 mg/ml Geneticin® (Invitrogen), and 10 ng/ml recombinant murine IL-3 (Peprotech, Inc.).

The ChemoTx® System (Neuro Probe Inc., 16008 Industrial Drive Gaithersburg, Md. 20877, USA) 96-well plate with a 5-micron pore size were used for chemotaxis (Chemo-TX # 106-5). Recombinant murine CXCL13 (R&D systems, cat #470-BC-025/CF, 100 nM) and anti-murine CXCL13 mAB (ranging from 30 ug/ml-0.03 ug/ml) were suspended in RPMI 1640 medium (Invitrogen) with 0.1% BSA (Invitrogen) in a final volume of 30 ul and added to the bottom chamber. Antibodies and chemokines were pre-incubated for 30 min at room temperature.

BA/F3/mCXCR5 cells (1×10⁵ cells/well)) in RPMI 1640+0.1% BSA were added to the top of the membrane. The plate assembly was incubated at 37 degrees centigrade with 5% CO2 for 2 hours. After incubation, residual cells were washed from the top of the membrane using DPBS (Invitrogen) and the plate was centrifuged at 1000 rpm for 1 minute. After centrifugation, the plate was disassembled and the bottom plate was placed at −80° C. for 10 minutes to lyse the migrated cells. Cells were quantified using the CyQUANT® Cell Proliferation Assay Kit (Invitrogen).

Raw data was analyzed using GraphPad Prism® 4 software (GraphPad Software, Inc.). IC50 values for the chemotaxis neutralizing antibodies were calculated using the non-linear regression and Kb values calculated using a modified Cheng-Prusoff equation (Leff and Dougall, Trends Pharmacol. Sci. 14 (4), 110-2 (1993)).

### Example 1.2

### Generation of Anti Mouse CXCL13 Monoclonal Antibodies

Anti mouse CXCL13 mouse monoclonal antibodies were obtained as follows: CXCL13 knockout mice (Ansel, K. M., V. N. Ngo, et al. (2000) Nature 406, p. 309). were immunized subcutaneously with 25 ug of MCXCL13 (R & D Systems) in Complete Freund's Adjuvant (Sigma). At three week intervals, mice were boosted with 25 ug of mCXCL13 in Incomplete Freund's Advjuvant (Sigma), subcutaneously for a total of three boosts. Four days prior to fusion, mice were boosted intravenously with 5-10 ug of antigen in saline.

### Example 1.2.B

### Generation of Hybridoma

To generate hybridomas, splenocytes obtained from the immunized mice described in Example 1.2.A were fused with SP2/O—Ag-14 cells at a ratio of 5:1 according to the established method described in Kohler, G. and Milstein 1975, Nature, 256:495. Fusion products were plated in selection media containing azaserine and hypoxanthine in 96-well plates at a density of 2.5×10⁶ spleen cells per well. Seven to ten days post fusion, macroscopic hybridoma colonies were observed. Supernatant from each well containing hybridoma colonies was tested by ELISA for the presence of antibody to mouse CXCL13 (as described in Example 1.1.A.1). Monoclonal antibodies were purified from supernatants displaying anti-mouse CXCL13-specific activity and tested for the ability to neutralize mouse CXCL13 in the FLIPR and Chemotaxis Assays (as described in Example 1.1.C).

### Example 1.2.C

### Identification and Characterization of Anti Mouse CXCL13 monoclonal Antibodies

Hybridomas producing antibodies that bound mouse CXCL13, generated according to Examples 1.2.B and 1.2.C, and capable of binding mouse CXCL13 specifically and particularly those with IC₅₀ values in the ELISA assay of 12 nM or less than 12 nM were scaled up and cloned by limiting dilution.

Hybridoma cells were expanded into media containing 10% low IgG fetal bovine serum (Hyclone #SH30151, Logan, Utah.). On average, 250 mL of each hybridoma supernatant (derived from a clonal population) was harvested, concentrated and purified by protein A affinity chromatography, as described in Harlow, E. and Lane, D. 1988 “Antibodies: A Laboratory Manual”. The ability of purified mabs to inhibit mouse CXCL13 activity was determined using the Calcium Flux (FLIPR) and/or Chemotaxis assays as described in Examples 1.1.C 2 and 1.1.C3. Table 4 shows IC₅₀ values from the assays for # monoclonal antibodies.

TABLE 4
Neutralization of CXCL13 by anti CXCL13
Murine Monoclonal Antibodies

| Murine | Average EC₅₀ | | Average Kb (nM) |
|---|---|---|---|
| Monoclonal | (nM) ELISA | Average Kb (nM) | Calcium Flux |
| Antibody | Assay | Chemotaxis Assay | (FLIPR) Assay |
| 5D11 | 0.03 | 18.9 | |
| 2C4 | 0.02 | 23.0 | 65.8 |
| 6A2 | 0.02 | 8.12 | 94.5 |
| 4F2 | 0.02 | 16 (n = 1) | |
| 7C7 | 0.02 | | |
| 8H6 | 0.03 | | |
| 9C10 | 0.01 | | |
| 3B5 | 0.04 | | |

The binding affinities of the monoclonal antibodies to recombinant purified mouse CXCL13 were determined using surface plasmon resonance (Biacore®) measurement as described in Example 1.1.B. Table 5 shows the affinity of the eight monoclonal antibodies described above for mouse CXCL 13.

TABLE 5
Affinity of anti CXCL13 Murine Monoclonal
Antibodies for Mouse CXCL13

| | kₒₙ | k_{off} | K_{D} |
|---|---|---|---|
| Name | (1/M · s) | (1/s) | (nM) |
| 1D4 | 2.5 × 10⁵ | 2.9 × 10⁻⁵ | 0.12 |

### Example 1.2.D Determination of the Amino Acid Sequences of the Variable Regions for Each Murine Anti-Murine CXCL13 mAb

For each amino acid sequence determination, approximately 10×10⁶ hybridoma cells were collected by centrifugation and processed to isolate total RNA by the RNAeasy Miniprep Kit (Qiagen, Valencia, Calif.) following manufacturer's instructions. Total RNA was subjected to first strand cDNA synthesis by the SuperScript First-Strand Synthesis System and random hexamer oligonucleotide primers per kit instructions (Invitrogen, Carlsbad, Calif.). The first strand cDNA product was then amplified by PCR with primers designed for amplification of murine immunoglobulin variable regions (primer sequences essentially as shown in the Ig-Primer Sets, Novagen, Madison, Wis.). Successful amplified PCR products were identified by agarose gel electrophoresis and sequences were determined directly from the PCR products. The cDNA sequences for the XW25-2C4, -3B5, and -5D11 MAbs were further confirmed by subcloning the amplified cDNA products into the pCR2.1-TOPO vector (Invitrogen, Carlsbad, Calif.) and transformed into TOP10F′ competent E. coli (Invitrogen, Carlsbad, Calif.). Colony PCR was performed on the transformants to identify clones containing insert and the amplified products were sequenced.

### Example 1.3 In Vivo Assays to Demonstrate Inhibition of Mouse CXCL13 Activity

Inhibition of mouse CXCL13 activity by anti-mouse CXCL13 monoclonal antibodies was measured by analyzing responses to thymus-dependent antigen and reduction of B1 cell number in the peritoneal cavity. In response to immunization with hapten-protein carrier conjugate mice form germinal centers and mount a T-cell mediated antibody response, measured by ELISPOT analysis, specific to the hapten.

### A. Thymus Dependent Antigen Immunization

3-month-old NZBW F1 mice were treated with anti-CXCL13 monoclonal antibody 2C4 2 times per week at varying concentrations (30, 10, 3, 1, 0.1 mpk) starting one day prior to immunization with hapten-protein carrier conjugate. The hapten-protein conjugate, NP-SRBC, was produced by incubating nitrophenyl acetic acid (NP; Biosearch Technologies) at a concentration of 1 mg/ml with a 10% solution of sheep red blood cells (SRBC; Innovative Research). The NZBW F1 mice were then immunized with 100 ul of NP-SRBC conjugate solution by intra-peritoneal injection. Injection of 2C4 continued 2 times per week for the duration of the study. On day 35 following immunization, the mice were boosted with a second intra-peritoneal injection of NP-SRBC. On day 41 the mice were sacrificed, serum collected by cardiac puncture, and spleen and peritoneal lavage samples collected.

B. ELISPOT Analysis of High Affinity anti-NP Antibody Secreting Cells

Falcon Immulon 2HB 96 well plates (Fischer) were coated with 2 mg/ml of NP₃-BSA or NP₁₂-BSA (Biosearch Technologies) and blocked with 3% BSA and 5% Goat serum in PBS. Single cell suspensions were produced from spleen and peritoneal lavage samples and red blood cells were lysed using Gey's Solution. Live cells were counted using Trypan Blue exclusion using a hemocytometer. Cells were added to the plates at 2×10⁶ cells per well and centrifuged at 1000 rpm for 5 min. The cells were incubated at 37° C. for 2 hours and then biotinylated anti-murine IgG was added to the wells and the cells continued incubation for 16 hours 37° C. Plates were washed and then Streptavidin-AP was added to the plates and allow to incubate at room temperature for 1 hour. The plates were then washed again and 50 ul of 1 mg/ml BCIP (Sigma) in AMP Buffer (0.75 mM MgCl₂ (Sigma) 0.01% Triton x405 (Sigma), 9.58% 2-amino-2-methyl-1-propanol (Sigma), pH 10.25 was added to each well. The spots that developed were read using an automated plate reader and the data statistically analyzed using Graphpad Prism 4. High affinity antibodies were determined by calculating the ratio of antibody secreting cells formed the bind to NP₃-BSA or NP₁₂-BSA.

Anti-murine CXCL13 treatment results in a 50±9.2% reduction in the production of high affinity secreting antibody cells at a dose of 30 mpk of 2C4 (Student's t test p<0.05).

### C. Flow Cytometry Analysis of Spleen and Peritoneal Lavage.

Single cell suspensions were produced from spleen and peritoneal lavage samples and red blood cells were lysed using Gey's Solution. Total cell counts were performed by Trypan Blue exclusion using a hemocytometer. Cells were then incubated with anti-CD16/32 antibody (BD Biosciences) to block non-specific FC receptor binding. Spleen cells were then assessed for germinal center formation by incubation with anti-GL7-FITC and anti-B220-APC antibodies (BD Biosciences) defining germinal center B cells as GL7⁺B220⁺. Peritoneal lavage cells were then assessed for B1 cell numbers by incubation with anti-CD5-PECy5 and anti-IgM-APC antibodies (BD Biosciences) defining B1 cells as CD5⁺IgM⁺.

Treatment of mice with anti-mouse CXCL13 causes a reduction in germinal center cell number in the spleen with an ED₅₀ of 3.736 mpk. Anti-mouse CXCL13 treatment also results in a reduction in peritoneal B1 cell number with an ED₅₀ of 29.27 mpk.

### D. Measurement of Serum 2C4 Antibody Levels

2C4 serum levels were measured by ELISA. 96 well plates were coated with 100 ul of 5 ug/ml polyclonal anti-mouse CXCL13 antibody (R&D) overnight at 4° C. Plates were then washed and incubated with Superblock (Pierce) overnight at 4° C. Plates were washed and incubated with 5 ug/ml of murine CXCL13 for 1 hour at room temperature. Plates were washed again and incubated with 2C4 containing serum samples, a standard curve of serial dilutions of 2C4, or negative control for 1 hour at room temperature. Plates were washed and incubated with goat anti-mouse IgG-HRP conjugate for 1 hour at room temperature. After washing, 100 ul of TMB substrate was added to the plates, the reaction stopped with H₂SO₄, and absorbance read at 450 nm using a spectrophotometer. Serum 2C4 levels were calculated using the standard curve of know dilutions of 2C4. Table 6 summarizes the serum 2C4 levels observed during anti-CXCL13 antibody treatment.

TABLE 6
2C4 Levels in anti-CXCL13 Treated Mice Following
Thymus Dependent Antigen Immunization.

| Time | Dose (mpk) | | | | |
|---|---|---|---|---|---|
| (days) | 30 | 10 | 3 | 1 | 0.1 |
| 3 | 152.9 ± 47.8 | 41.9 ± 11.4 | 9.5 ± 1.8 | 2.9 ± 0.6 | — |
| 4 | 325.2 ± 112.8 | 101.3 ± 47.7 | 22.8 ± 3.7 | — | — |
| 14 | 233.9 ± 65.1 | 45.7 ± 22.7 | 7.7 ± 7.7 | 3.0 ± 0.7 | 1.7 ± 1.4 |
| 27 | 593.9 ± 376.4 | 55.6 ± 61.5 | 15.5 ± 7.8 | 21.7 ± 11.4 | 9.2 ± 3.6 |
| 39 | 579.2 ± 240.0 | 101.2 ± 56.3 | 34.7 ± 10.0 | 27.8 ± 10.1 | 9.2 ± 3.8 |
| 41 | 724.1 ± 442.7 | 61.2 ± 80.1 | 28.7 ± 16.6 | 29.8 ± 9.2 | 10.8 ± 1.7 |

The present invention incorporates by reference in their entirety techniques well known in the field of molecular biology. These techniques include, but are not limited to, techniques described in the following publications:
Ausubel, F. M. et al. eds., Short Protocols In Molecular Biology (4th Ed. 1999) John Wiley & Sons, NY. (ISBN 0471-32938-X).
Ansel, K. M., V. N. Ngo, et al. A chemokine-driven positive feedback loop organizes lymphoid follicles (2000) Nature 406(6793):p. 309.
Lu and Weiner eds., Cloning and Expression Vectors for Gene Function Analysis (2001) BioTechniques Press. Westborough, Mass. 298 pp. (ISBN¹-881299-21-X). Kontermann and Dubel eds., Antibody Engineering (2001) Springer-Verlag. New York. 790 pp. (ISBN 3-54041354-5).
Old, R. W. & S. B. Primrose, Principles of Gene Manipulation: An Introduction To Genetic Engineering (3d Ed. 1985) Blackwell Scientific Publications, Boston. Studies in Microbiology; V.2:409 pp. (ISBN 0-632-01318-4).
Sambrook, J. et al. eds., Molecular Cloning: A Laboratory Manual (2d Ed. 1989) Cold Spring Harbor Laboratory Press, NY. Vols. 1-3. (ISBN 0-87969-309-6).
Winnacker, E. L. From Genes To Clones: Introduction To Gene Technology (1987) VCH Publishers, NY (translated by Horst Ibelgaufts). 634 pp. (ISBN 0-89573-6144).

Although a number of embodiments and features have been described above, it will be understood by those skilled in the art that modifications and variations of the described embodiments and features may be made without departing from the present disclosure or the invention as defined in the appended claims. Each of the publications mentioned herein is incorporated by reference.

We claim:

1. An isolated binding protein, or fragment thereof, that binds mouse CXCL13.

2. The isolated binding protein of claim 1, wherein the isolated binding protein, or fragment thereof, is capable of modulating a biological function of mouse CXCL13.

3. The isolated binding protein of claim 1, wherein the isolated binding protein, or fragment thereof, is capable of neutralizing mouse CXCL13.

4. The isolated binding protein of claim 1, wherein the isolated binding protein, or fragment thereof, is capable of preventing the binding of mouse CXCL13 to the mouse CXCL13 receptor CXCL5.

5. The isolate binding protein of claim 1, wherein the isolated binding protein, or fragment thereof, binds mouse CXCL13 with an EC₅₀ selected from the group consisting of less than about 12.0 nM using an ELISA test.

6. The isolate binding protein of claim 1, wherein the isolated binding protein, or fragment thereof inhibits calcium influx induced by said mouse CXCL13 to the mouse CXCL13 receptor CXCL5 in a CXCL5 receptor mediated FLIPR assay with an average Kb of about 65 to about 95.

7. The isolated binding protein of claim 1, wherein the isolated binding protein has been affinity maturated.

8. An isolated binding protein comprising an antigen binding domain, the isolated binding protein capable of binding mouse CXCL13, the antigen binding domain comprising at least one CDR comprising an amino acid sequence selected from the group consisting of SEQ ID NOs: 6, 7, 8, 10, 11, 12, 14, 15, 16, 18, 19, 20, 22, 23, 24, 26, 27, 28, 30, 31, 32, 34, 35, 36, 38, 39, 40, 42, 43, 44, 46, 47, 48, 50, 51, 52, 54, 55, 56, 58, 59, 60, 62, 63, 64, 66, 67, and 68.

9. An isolated binding protein comprising an antigen binding domain, wherein the binding protein is capable of binding mouse CXCL13, and wherein the antigen binding domain comprises at least one VH region comprising an amino acid sequence selected from the group consisting of SEQ ID NOs: 5, 13, 21, 29, 37, 45, 53, and 61.

10. An isolated binding protein comprising an antigen binding domain, wherein the binding protein is capable of binding mouse CXCL13, and wherein the antigen binding domain comprises at least one VL region comprising an amino acid sequence selected from the group consisting of SEQ ID NOs: 9, 17, 25, 33, 41, 49, 57, and 65.

11. The isolated binding protein of claim 8, wherein said binding protein comprises at least 3 CDRs.

12. The isolated binding protein of claim 11, wherein said at least 3 CDRs are selected from a VH CDR set selected from the group consisting of SEQ ID NOs: 6, 7, and 8; SEQ ID NOs: 14, 15, and 16; SEQ ID NOs: 22, 23, and 24; SEQ ID NOs: 30, 31, and 32; SEQ ID NOs: 38, 39, and 40; and SEQ ID NOs: 46, 47, and 48; SEQ ID NOs: 54, 55, and 56; and SEQ ID NOs: 62, 63, and 64.

13. The isolated binding protein of claim 13, wherein said at least 3 CDRs are selected from a VL CDR set selected from the group consisting of SEQ ID NOs: 10, 11, and 12; SEQ ID NOs: 18, 19, and 20; SEQ ID NOs: 26, 27, and 28; SEQ ID NOs: 34, 35, and 36; SEQ ID NOs: 42, 43, and 44; and SEQ ID NOs: 50, 51, and 52; SEQ ID NOs: 58, 59, and 60; and SEQ ID NOs: 66, 67, and 68.

14. The isolated binding protein of claim 10, wherein said binding protein comprises at least two variable domain CDR sets.

15. The isolated binding protein of claim 16, wherein said at least two variable domain CDR sets are selected from a group consisting of: SEQ ID NOs: 6, 7, 8 and SEQ ID NOs: 10, 11, 12; SEQ ID NOs: 14, 15 16 and SEQ ID NOs: 18, 19, 20; SEQ ID NOs: 22, 23, 24 and SEQ ID NOs: 26, 27, 28; SEQ ID NOs: 30, 31, 32 and SEQ ID NOs: 34, 35, 36; SEQ ID NOs: 38, 39, 40 and SEQ ID NOs: 42, 43, 44; SEQ ID NOs: 46, 47, 48 and SEQ ID NOs: 50, 51, 52; SEQ ID NOs: 54, 55, 56 and SEQ ID NOs: 58, 59, 60; and SEQ ID NOs: 62, 63, 64 and SEQ ID NOs: 66, 67, 68.

16. The isolated binding protein of claim 1, wherein said binding protein comprises two variable domains, wherein said two variable domains have amino acid sequences selected from the group consisting of SEQ ID NO:5 and SEQ ID NO:9; SEQ ID NO:13 and SEQ ID NO:17; SEQ ID NO:21 and SEQ ID NO:25; SEQ ID NO:29 and SEQ ID NO:33; SEQ ID NO:37 and SEQ ID NO:41; SEQ ID NO:45 and SEQ ID NO:49; SEQ ID NO:53 and SEQ ID NO:57; and SEQ ID NO:61 and SEQ ID NO:65.

17. A isolated binding protein that binds a protein comprising a sequence selected from the group consisting of SEQ ID NO: 3 and SEQ ID NO:4.

18. An antibody construct comprising the binding protein of claim 1, said antibody construct further comprising a linker polypeptide or an immunoglobulin constant domain.

19. The antibody construct of claim 18, wherein said binding protein is selected from the group consisting of an immunoglobulin molecule, a disulfide linked Fv, a monoclonal antibody, a scFv, a chimeric antibody, a single domain antibody, a CDR-grafted antibody, a diabody, a humanized antibody, a multispecific antibody, a Fab, a dual specific antibody, and a Fab′, a bispecific antibody, a F(ab′)2, and a Fv.

20. The antibody construct of claim 18, wherein said binding protein comprises a heavy chain immunoglobulin constant domain selected from the group consisting of a human IgM constant domain, a human IgG4 constant domain, a human IgG1 constant domain, a human IgE constant domain, a human IgG2 constant domain, and a human IgG3 constant domain, a human IgA constant domain.

21. The antibody construct of claim 18, wherein said binding protein possesses a human glycosylation pattern.

22. The antibody construct of claim 18, wherein said antibody construct is a crystallized antibody construct.

23. The antibody construct of claim 22, wherein said crystallized antibody construct is a carrier-free pharmaceutical controlled release crystallized antibody construct.

24. The antibody construct of claim 18, wherein said antibody construct has a greater half life in vivo than the soluble counterpart of said antibody construct.

25. The antibody construct of claim 18, wherein said antibody construct retains biological activity.

26. An antibody conjugate comprising an antibody construct of claim 18, said antibody conjugate further comprising an agent selected from the group consisting of; an immunoadhension molecule, an imaging agent, a therapeutic agent, and a cytotoxic agent.

27. The antibody conjugate of claim 26, wherein said agent is an imaging agent selected from the group consisting of a radiolabel, an enzyme, a fluorescent label, a luminescent label, a bioluminescent label, a magnetic label, and biotin.

28. The antibody conjugate of claim 27, wherein said imaging agent is a radiolabel selected from the group consisting of: ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm.

29. The antibody conjugate of claim 26, wherein said agent is a therapeutic or cytotoxic agent selected from the group consisting of; an anti-metabolite, an alkylating agent, an antibiotic, a growth factor, a cytokine, an anti-angiogenic agent, an anti-mitotic agent, an anthracycline, toxin, and an apoptotic agent.

30. The isolated binding protein of claim 1, wherein said binding protein is a crystallized binding protein.

31. An isolated nucleic acid encoding a binding protein amino acid sequence of claim 1.

32. An isolated nucleic acid encoding an antibody construct comprising an amino acid sequence of claim 31.

33. A vector comprising an isolated nucleic acid of claim 31.

34. The vector of claim 33, wherein said vector is selected from the group consisting of pcDNA, pTT, pTT3, pEFBOS, pBV, pJV, and pBJ.

35. A host cell comprising a vector of claim 33.

36. The host cell of claim 35, wherein said host cell is a prokaryotic cell.

37. The host cell of claim 36, wherein said host cell is E. coli.

38. The host cell of claim 35, wherein said host cell is a eukaryotic cell.

39. The host cell of claim 38, wherein said eukaryotic cell is selected from the group consisting of protist cell, animal cell, plant cell and fungal cell.

40. The host cell of claim 38, wherein said eukaryotic cell is an animal cell selected from the group consisting of; a mammalian cell, an avian cell, and an insect cell.

41. The host cell of claim 38, wherein said host cell is a CHO cell.

42. The host cell of claim 38, wherein said host cell is COS.

43. The host cell of claim 38, wherein said host cell is a yeast cell.

44. The host cell of claim 43, wherein said yeast cell is Saccharomyces cerevisiae.

45. The host cell of claim 38, wherein said host cell is an insect Sf9 cell.

46. A method of producing a protein capable of binding mouse CXCL13, the method comprising the steps of culturing a host cell of claim 35 in culture medium under conditions sufficient to produce a binding protein capable of binding mouse CXCL13.

47. A protein produced according to the method of claim 46.

48. A method for reducing CXCL13 activity in a mammal, comprising contacting CXCL13 with the binding protein of claim 1 such that CXCL13 activity is reduced.

49. A method for reducing CXCL13 activity in a mammal suffering from a disorder in which CXCL13 activity is detrimental, comprising administering to the mammal the binding protein of claim 1 such that CXCL13 activity in the mammal is reduced.

50. A method for treating a mammal for a disease or a disorder in which CXCL13 activity is detrimental by administering to the mammal the binding protein of claim 1 such that treatment is achieved.

51. The method of claim 50, wherein said disorder is selected from the group consisting of respiratory disorders; asthma; allergic and nonallergic asthma; asthma due to infection; asthma due to infection with respiratory syncytial virus (RSV); chronic obstructive pulmonary disease (COPD); other conditions involving airway inflammation; eosinophilia; fibrosis and excess mucus production; cystic fibrosis; pulmonary fibrosis; atopic disorders; atopic dermatitis; urticaria; eczema; allergic rhinitis; and allergic enterogastritis; inflammatory and/or autoimmune conditions of the skin; inflammatory and/or autoimmune conditions of gastrointestinal organs; inflammatory bowel diseases (IBD); ulcerative colitis; Crohn's disease; inflammatory and/or autoimmune conditions of the liver; liver cirrhosis; liver fibrosis; liver fibrosis caused by hepatitis B and/or C virus; scleroderma; tumors or cancers; hepatocellular carcinoma; glioblastoma; lymphoma; Hodgkin's lymphoma; viral infections; HTLV-1 infection (e.g., from HTLV-1); suppression of expression of protective type I immune responses, and suppression of expression of protective type 1 immune responses during vaccination.

52. The method of claim 50, wherein said disorder is selected from the group consisting of rheumatoid arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjörgren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma), Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aordic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, Burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chromic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetes, diabetes mellitus, diabetic ateriosclerotic disease, Diffuse Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, epstein-barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallerrorden-Spatz disease, hashimoto's thyroiditis, hay fever, heart transplant rejection, hemachromatosis, hemodialysis, hemolytic uremic syndrome/thrombolytic thrombocytopenic purpura, hemorrhage, hepatitis (A), His bundle arrythmias, HIV infection/HIV neuropathy, Hodgkin's disease, hyperkinetic movement disorders, hypersensitivity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic neuritis, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphederma, malaria, malignamt Lymphoma, malignant histiocytosis, malignant melanoma, meningitis, meningococcemia, metabolic/idiopathic, migraine headache, mitochondrial multi.system disorder, mixed connective tissue disease, monoclonal gammopathy, multiple myeloma, multiple systems degenerations (Mencel Dejerine-Thomas Shi-Drager and Machado-Joseph), myasthenia gravis, mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occulsive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherlosclerotic disease, peripheral vascular disorders, peritonitis, pernicious anemia, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, Progressive supranucleo Palsy, primary pulmonary hypertension, radiation therapy, Raynaud's phenomenon and disease, Raynoud's disease, Refsum's disease, regular narrow QRS tachycardia, renovascular hypertension, reperfusion injury, restrictive cardiomyopathy, sarcomas, scleroderma, senile chorea, Senile Dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, Subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, Acute coronary syndromes, Acute Idiopathic Polyneuritis, Acute Inflammatory Demyelinating Polyradiculoneuropathy, Acute ischemia, Adult Still's Disease, Alopecia greata, Anaphylaxis, Anti-Phospholipid Antibody Syndrome, Aplastic anemia, Arteriosclerosis, Atopic eczema, Atopic dermatitis, Autoimmune dermatitis, Autoimmune disorder associated with Streptococcus infection, Autoimmune Enteropathy, Autoimmune hearingloss, Autoimmune Lymphoproliferative Syndrome (ALPS), Autoimmune myocarditis, Autoimmune premature ovarian failure, Blepharitis, Bronchiectasis, Bullous pemphigoid, Cardiovascular Disease, Catastrophic Antiphospholipid Syndrome, Celiac Disease, Cervical Spondylosis, Chronic ischemia, Cicatricial pemphigoid, Clinically isolated Syndrome (CIS) with Risk for Multiple Sclerosis, Conjunctivitis, Childhood Onset Psychiatric Disorder, Chronic obstructive pulmonary disease (COPD), Dacryocystitis, dermatomyositis, Diabetic retinopathy, Diabetes mellitus, Disk herniation, Disk prolaps, Drug induced immune hemolytic anemia, Endocarditis, Endometriosis, endophthalmitis, Episcleritis, Erythema multiforme, erythema multiforme major, Gestational pemphigoid, Guillain-Barré Syndrome (GBS), Hay Fever, Hughes Syndrome, Idiopathic Parkinson's Disease, idiopathic interstitial pneumonia, IgE-mediated Allergy, Immune hemolytic anemia, Inclusion Body Myositis, Infectious ocular inflammatory disease, Inflammatory demyelinating disease, Inflammatory heart disease, Inflammatory kidney disease, IPF/UIP, Iritis, Keratitis, Keratojuntivitis sicca, Kussmaul disease or Kussmaul-Meier Disease, Landry's Paralysis, Langerhan's Cell Histiocytosis, Livedo reticularis, Macular Degeneration, Microscopic Polyangiitis, Morbus Bechterev, Motor Neuron Disorders, Mucous membrane pemphigoid, Multiple Organ failure, Myasthenia Gravis, Myelodysplastic Syndrome, Myocarditis, Nerve Root Disorders, Neuropathy, Non-A Non-B Hepatitis, Optic Neuritis, Osteolysis, Pauciarticular JRA, peripheral artery occlusive disease (PAOD), peripheral vascular disease (PVD), peripheral artery disease (PAD), Phlebitis, Polyarteritis nodosa (or periarteritis nodosa), Polychondritis, Polymyalgia Rheumatica, Poliosis, Polyarticular JRA, Polyendocrine Deficiency Syndrome, Polymyositis, polymyalgia rheumatica (PMR), Post-Pump Syndrome, primary parkinsonism, Prostatitis, Pure red cell aplasia, Primary Adrenal Insufficiency, Recurrent Neuromyelitis Optica, Restenosis, Rheumatic heart disease, SAPHO (synovitis, acne, pustulosis, hyperostosis, and osteitis), Scleroderma, Secondary Amyloidosis, Shock lung, Scleritis, Sciatica, Secondary Adrenal Insufficiency, Silicone associated connective tissue disease, Sneddon-Wilkinson Dermatosis, spondilitis ankylosans, Stevens-Johnson Syndrome (SJS), Systemic inflammatory response syndrome, Temporal arteritis, toxoplasmic retinitis, toxic epidermal necrolysis, Transverse myelitis, TRAPS (Tumor Necrosis Factor Receptor, Type I allergic reaction, Type II Diabetes, Urticaria, Usual interstitial pneumonia (UIP), Vasculitis, Vernal conjunctivitis, viral retinitis, Vogt-Koyanagi-Harada syndrome (VKH syndrome), Wet macular degeneration, and Wound healing.

53. A method of treating a mammal suffering from a disorder in which CXCL13 is detrimental, the method comprising the step of administering the binding protein of claim 1 before, concurrent, or after the administration of a second agent, wherein the second agent is selected from the group consisting of inhaled steroids; beta-agonists; short-acting or long-acting beta-agonists; antagonists of leukotrienes or leukotriene receptors; ADVAIR; IgE inhibitors; anti-IgE antibodies; XOLAIR; phosphodiesterase inhibitors; PDE4 inhibitors; xanthines; anticholinergic drugs; mast cell-stabilizing agents; Cromolyn; IL-4 inhibitors; IL-5 inhibitors; eotaxin/CCR3 inhibitors; antagonists of histamine or its receptors including H1, H2, H3, and H4; antagonists of prostaglandin D or its receptors DP1 and CRTH2; TNF antagonists; a soluble fragment of a TNF receptor; ENBREL; TNF enzyme antagonists; TNF converting enzyme (TACE) inhibitors; muscarinic receptor antagonists; TGF-beta antagonists; interferon gamma; perfenidone; chemotherapeutic agents, methotrexate; leflunomide; sirolimus (rapamycin) or an analog thereof, CCI-779; COX2 or cPLA2 inhibitors; NSAIDs; immunomodulators; p38 inhibitors; TPL-2, MK-2 and NFkB inhibitors; budenoside; epidermal growth factor; corticosteroids; cyclosporine; sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β antibodies; anti-IL-6 antibodies; growth factors; elastase inhibitors; pyridinyl-imidazole compounds; antibodies or agonists of TNF, LT, IL-1, IL-2, IL-3, IL-4, IL-5, IL-6, IL-7, IL-8, IL-9, IL-10, IL-11, IL-12, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, EMAP-II, GM-CSF, FGF, or PDGF; antibodies of CD2, CD3, CD4, CD8, CD25, CD28, CD30, CD40, CD45, CD69, CD90 or their ligands; FK506; rapamycin; mycophenolate mofetil; ibuprofen; prednisolone; phosphodiesterase inhibitors; adenosine agonists; antithrombotic agents; complement inhibitors; adrenergic agents; IRAK, NIK, IKK, p38, or MAP kinase inhibitors; IL-11 converting enzyme inhibitors; TNFα converting enzyme inhibitors; T-cell signaling inhibitors; metalloproteinase inhibitors; 6-mercaptopurines; angiotensin converting enzyme inhibitors; soluble cytokine receptors; soluble p55 TNF receptor; soluble p75 TNF receptor; sIL-IR1; sIL-IRII; sIL-6R; anti-inflammatory cytokines; IL-4; IL-10; IL-11; and TGFβ.

54. The method of claim 53, wherein said administering to the subject is by at least one mode selected from parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal.

55. An isolated antibody, or antigen binding fragment thereof, wherein said antibody, or antigen binding fragment thereof binds mouse CXCL13 and inhibits the binding of said CXCL13 to CXCR5 receptor in a cell surface-based receptor binding assay with an IC₅₀ selected from the group consisting of less than about 12.0 nM using an ELISA test.

56. The isolate binding protein of claim 55, wherein the isolated binding protein, or fragment thereof inhibits calcium influx induced by said mouse CXCL13 to the mouse CXCL13 receptor CXCL5 in a CXCL5 receptor mediated FLIPR assay with an average Kb of about 65 to about 95.

57. The antibody of claim 55, wherein said antibody is selected from the group consisting of 5D11, 2C4, 6A2, 4F2, 7C7, 8H6, 9C10, and 3B5.

58. An isolated antibody, or antigen binding fragment thereof, wherein said antibody or antigen binding fragment thereof, binds mouse CXCL13 with binding characteristics selected from the group consisting of:
a. an on rate constant (kₒₙ) between about 1.0×10⁶M⁻¹ s⁻¹ to about 3×10⁶M⁻¹ s⁻¹;
b. an off rate constant (k_{off}) of about 3.0×10⁻⁴ s⁻¹ to about 3.0×10⁻⁶ s⁻¹; as measured by surface plasmon resonance; and
c. a dissociation constant (K_{D}) of about 2 pM to about 13 pM.

59. The antibody, or antigen binding fragment thereof, of claim 58, where in said antibody, or antigen binding fragment thereof has a dissociation constant (K_{D}) to mouse CXCL13 selected from the group consisting of about 2.0 pM, about 3.0 pM, and about 13.0 pM.

60. The antibody, or antigen binding fragment thereof, of claim 58, wherein said antibody, or antigen binding fragment thereof, is capable of modulating a biological function of mouse CXCL13.

61. The antibody, or antigen binding fragment thereof, of claim 58, wherein said antibody, or antigen binding fragment thereof, is capable of neutralizing mouse CXCL13.

62. A method of generating an antibody, or fragment thereof, that binds to mouse CXCL13, the method comprising the steps of immunizing a non-human animal with CXCL13; collecting a body fluid or organ comprising an anti-CXCL13 antibody; and isolating said anti-CXCL13 antibody.
